SEQUENCE LISTING (246 sequences)

SEQ ID NO: 1 - protein, 22 aa, Synthetic construct
YSQQSSSYGQQNPSYDSVRRGA

SEQ ID NO: 2 - protein, 22 aa, Synthetic construct
YSQQSSSYGQQNPSYDQVRRGA

SEQ ID NO: 3 - protein, 20 aa, Synthetic construct
YQSSSYGQQDPSYDSVRRGA

SEQ ID NO: 4 - protein, 20 aa, Synthetic construct
YQSSSYGQQNPSYDSVRRGA

SEQ ID NO: 5 - protein, 24 aa, Synthetic construct
QYSQQSSSYGQQNPSYDSVRRGAW

SEQ ID NO: 6 - protein, 22 aa, Synthetic construct
QYSQQSSSYGQQNPSYDSVRRG

SEQ ID NO: 7 - protein, 22 aa, Synthetic construct
SQQSSSYGQQNPSYDSVRRGAW

SEQ ID NO: 8 - protein, 23 aa, Synthetic construct
YSQQSSSYGQQNPSYDSVRRGAW

SEQ ID NO: 9 - protein, 23 aa, Synthetic construct
YSQQSSSYGQQNPSYDSVRRGAW

SEQ ID NO: 10 - protein, 30 aa, Synthetic construct
YSQAPSQYSQQSSSYGQQNPSYDSVRRGAW

SEQ ID NO: 11 - protein, 17 aa, Synthetic construct
QSSSYGQQSSLLAYNTT

SEQ ID NO: 12 - protein, 23 aa, Synthetic construct
QYSQQSSSYGQQSSLLAYNTTSH

SEQ ID NO: 13 - protein, 24 aa, Synthetic construct
QYSQQSSSYGQQSSLLAYNTTSHT

SEQ ID NO: 14 - protein, 20 aa, Synthetic construct
SQQSSSYGQQSSLLAYNTTS

SEQ ID NO: 15 - protein, 17 aa, Synthetic construct
QSSSYGQQSSLLAYNTT

SEQ ID NO: 16 - protein, 24 aa, Synthetic construct
QYSQQSSSYGQQNPYQILGPTSSR

SEQ ID NO: 17 - protein, 20 aa, Synthetic construct
SQQSSSYGQQRSGQIQLWQF

SEQ ID NO: 18 - protein, 17 aa, Synthetic construct
DEGPDLDLGSLLAYNTT

SEQ ID NO: 19 - protein, 21 aa, Synthetic construct
PMDEGPDLDLGSLLAYNTTSH

SEQ ID NO: 20 - protein, 19 aa, Synthetic construct
PMDEGPDLDLDPSYDSVRR

SEQ ID NO: 21 - protein, 20 aa, Synthetic construct
PMDEGPDLDLDPSYDSVRRG

SEQ ID NO: 22 - protein, 18 aa, Synthetic construct
MDEGPDLDLDPSYDSVRR

SEQ ID NO: 23 - protein, 20 aa, Synthetic construct
GERGGFNKPGGPPLGGAQTI

SEQ ID NO: 24 - protein, 32 aa, Synthetic construct
GTEVNACVEFSSLIDQPVYPDVLASGRWGSSA

SEQ ID NO: 25 - protein, 22 aa, Synthetic construct
GPMDEGPDLDLGSLLAYNTTSH

SEQ ID NO: 26 - protein, 20 aa, Synthetic construct
SQQSSSYGQQNPVGDGLFKD

SEQ ID NO: 27 - protein, 16 aa, Synthetic construct
QSSSYGQQRGAAFIFP

SEQ ID NO: 28 - protein, 20 aa, Synthetic construct
SQQSSSYGQQNLPYEPPRRS

SEQ ID NO: 29 - protein, 20 aa, Synthetic construct
PMDEGPDLDLDLPYEPPRRS

SEQ ID NO: 30 - protein, 10 aa, Synthetic construct
SSYGQQNLPY

SEQ ID NO: 31 - protein, 11 aa, Synthetic construct
SSSYGQQNLPY

SEQ ID NO: 32 - protein, 10 aa, Synthetic construct
SSYGQQNLPY

SEQ ID NO: 33 - protein, 9 aa, Synthetic construct
SSSYGQQNL

SEQ ID NO: 34 - protein, 24 aa, Synthetic construct
QYSQQSSSYGQQNLPYEPPRRSAW

SEQ ID NO: 35 - protein, 15 aa, Synthetic construct
GGRGGMGKILKDLSS

SEQ ID NO: 36 - protein, 20 aa, Synthetic construct
GRGGGRGGMGKILKDLSSED

SEQ ID NO: 37 - protein, 20 aa, Synthetic construct
SQQSSSYGQQSEKPYQCDFK

SEQ ID NO: 38 - protein, 17 aa, Synthetic construct
QSSSYGQQSEKPYQCDF

SEQ ID NO: 39 - protein, 17 aa, Synthetic construct
QSSSYGQQSEKPYQCDF

SEQ ID NO: 40 - protein, 17 aa, Synthetic construct
RGGFNKPGGEKPYQCDF

SEQ ID NO: 41 - protein, 20 aa, Synthetic construct
GERGGFNKPGGEKPYQCDFK

SEQ ID NO: 42 - protein, 28 aa, Synthetic construct
GQEVEGLVVLSSHLSPPRLLSLPRAFLL

SEQ ID NO: 43 - protein, 22 aa, Synthetic construct
HMNPVSNGLSPQNSIRHNLSLH

SEQ ID NO: 44 - protein, 20 aa, Synthetic construct
PSDLASDNYWHKLEVTPVVA

SEQ ID NO: 45 - protein, 20 aa, Synthetic construct
TEQPPGAEAPSPWWGWGRGP

SEQ ID NO: 46 - protein, 20 aa, Synthetic construct
PDPTSPSSDSPWWGWGRGPQ

SEQ ID NO: 47 - protein, 20 aa, Synthetic construct
DPTSPSSDSGRTAVTGSQTA

SEQ ID NO: 48 - protein, 20 aa, Synthetic construct
PQQRPYGYDQIMPKKPAEEG

SEQ ID NO: 49 - protein, 16 aa, Synthetic construct
RTKVLGQSVMTFIMLR

SEQ ID NO: 50 - protein, 21 aa, Synthetic construct
SREKTEDKVNPLFIKVRGSGF

SEQ ID NO: 51 - protein, 18 aa, Synthetic construct
LSFGYVRPSVRLPTRCMT

SEQ ID NO: 52 - DNA, 66 bp, Synthetic construct
  source 1..66: mol_type = other DNA
atagccaacagagcagcagctacgggcagcagaacccttcttatgactcagtcagaagaggagctt

SEQ ID NO: 53 - DNA, 63 bp, Synthetic construct
  source 1..63: mol_type = other DNA
atagccaacagagcagcagctacgggcagcagaacccttcttatgactcagtcagaagagctt

SEQ ID NO: 54 - DNA, 63 bp, Synthetic construct
  source 1..63: mol_type = other DNA
atagccaacacagagcagcagctacgggcagcagaacccttcttatgactcagtcagaagctt

SEQ ID NO: 55 - DNA, 58 bp, Synthetic construct
  source 1..58: mol_type = other DNA
cagagcagcagctacgggcagcagaacccttcttatgactcagtcagaagaggagctt

SEQ ID NO: 56 - DNA, 65 bp, Synthetic construct
  source 1..65: mol_type = other DNA
atagccaacagagcagcagctacgggcagcagacccttcttatgactcagtcagaagaggagcat

SEQ ID NO: 57 - DNA, 66 bp, Synthetic construct
  source 1..66: mol_type = other DNA
atagccaacagagcagcagctacgggcagcagaacccttcttatgactcagtcagaagaggagcat

SEQ ID NO: 58 - DNA, 72 bp, Synthetic construct
  source 1..72: mol_type = other DNA
aatatagccaacagagcagcagctacgggcagcagaacccttcttatgactcagtcagaagaggagcttttg

SEQ ID NO: 59 - DNA, 72 bp, Synthetic construct
  source 1..72: mol_type = other DNA
aatatagccaacagagcagcagctacgggcagcagaacccttcttatgactcagtcagaagaggagcttggg

SEQ ID NO: 60 - DNA, 65 bp, Synthetic construct
  source 1..65: mol_type = other DNA
aatatagccaacagagcagcagctacgggcagcagacccttcttatgactcagtcagaagaggag

SEQ ID NO: 61 - DNA, 68 bp, Synthetic construct
  source 1..68: mol_type = other DNA
aatatagccaacagagcagcagctacgggcagcagacccttcttatgactcagtcagaagagcttggg

SEQ ID NO: 62 - DNA, 69 bp, Synthetic construct
  source 1..69: mol_type = other DNA
atagccaacagagcagcagctacgggcagcaaacccttcttatgactcagtcagaagaggagcttgggg

SEQ ID NO: 63 - DNA, 66 bp, Synthetic construct
  source 1..66: mol_type = other DNA
aatatagccaacagagcagcagctacgggcagcagaacccttcttatgactcagtcagaagaggag

SEQ ID NO: 64 - DNA, 68 bp, Synthetic construct
  source 1..68: mol_type = other DNA
aatatagccaacagagcagcagctacgggcagcagaacccttcttatgactcagtcagaagaggagct

SEQ ID NO: 65 - DNA, 68 bp, Synthetic construct; identical to SEQ ID NO: 64
  source 1..68: mol_type = other DNA

SEQ ID NO: 66 - DNA, 67 bp, Synthetic construct
  source 1..67: mol_type = other DNA
tatagccaacagagcagcagctacgggcagcagaacccttcttatgactcagtcagaagaggagcat

SEQ ID NO: 67 - DNA, 51 bp, Synthetic construct
  source 1..51: mol_type = other DNA
cagagcagcagctacgggcagcagagttcactgctggcctataatacaacc

SEQ ID NO: 68 - DNA, 72 bp, Synthetic construct
  source 1..72: mol_type = other DNA
aatatagccaacagagcagcagctacgggcagcagagttcactgctggcctataatacaacctcccacaccg

SEQ ID NO: 69 - DNA, 72 bp, Synthetic construct; identical to SEQ ID NO: 68
  source 1..72: mol_type = other DNA

SEQ ID NO: 70 - DNA, 72 bp, Synthetic construct
  source 1..72: mol_type = other DNA
aatataccaacagagcagcagctacgggcagcagagttcactgctggcctataatacaacctcccacaccga

SEQ ID NO: 71 - DNA, 60 bp, Synthetic construct
  source 1..60: mol_type = other DNA
gccaacagagcagcagctacgggcagcagagttcactgctggcctataatacaacctccc

SEQ ID NO: 72 - DNA, 61 bp, Synthetic construct
  source 1..61: mol_type = other DNA
tagccaacagagcagcagctacgggcagcagagttcactgctggcctataatacaacctcc

SEQ ID NO: 73 - DNA, 52 bp, Synthetic construct
  source 1..52: mol_type = other DNA
cagagcagcagctacgggcagcagagttcactgctggcctataatacaacct

SEQ ID NO: 74 - DNA, 60 bp, Synthetic construct; identical to SEQ ID NO: 71
  source 1..60: mol_type = other DNA

SEQ ID NO: 75 - DNA, 60 bp, Synthetic construct; identical to SEQ ID NO: 71
  source 1..60: mol_type = other DNA

SEQ ID NO: 76 - DNA, 60 bp, Synthetic construct
  source 1..60: mol_type = other DNA
agccaacagagcagcagctacgggcagcagagttcactgctggcctataatacaacctcc

SEQ ID NO: 77 - DNA, 57 bp, Synthetic construct
  source 1..57: mol_type = other DNA
aacagagcagcagctacgggcagcagagttcactgctggcctataatacaacctccc

SEQ ID NO: 78 - DNA, 60 bp, Synthetic construct; identical to SEQ ID NO: 71
  source 1..60: mol_type = other DNA

SEQ ID NO: 79 - DNA, 62 bp, Synthetic construct
  source 1..62: mol_type = other DNA
acagagcagcagctacgggcagcagaatccgtatcagatcctgggcccgaccagcagtcgcc

SEQ ID NO: 80 - DNA, 60 bp, Synthetic construct
  source 1..60: mol_type = other DNA
gccaacagagcagcagctacgggcagcagagaagcgggcagatccagctgtggcaattcc

SEQ ID NO: 81 - DNA, 51 bp, Synthetic construct
  source 1..51: mol_type = other DNA
gatgaaggaccagatcttgatctaggttcactgctggcctataatacaacc

SEQ ID NO: 82 - DNA, 60 bp, Synthetic construct
  source 1..60: mol_type = other DNA
ccatggatgaaggaccagatcttgatctaggttcactgctggcctataatacaacctccc

SEQ ID NO: 83 - DNA, 64 bp, Synthetic construct
  source 1..64: mol_type = other DNA
gacccatggatgaaggaccagatcttgatctaggttcactgctggcctataatacaacctccca

SEQ ID NO: 84 - DNA, 60 bp, Synthetic construct
  source 1..60: mol_type = other DNA
gaccagatcttgatctagacccttcttatgactcagtcagaagaggagcttggggcaata

SEQ ID NO: 85 - DNA, 59 bp, Synthetic construct
  source 1..59: mol_type = other DNA
ccatggatgaaggaccagatcttgatctaacccttcttatgactcagtcagaagaggag

SEQ ID NO: 86 - DNA, 56 bp, Synthetic construct
  source 1..56: mol_type = other DNA
tggatgaaggaccagatcttgatctagacccttcttatgactcagtcagaagagga

SEQ ID NO: 87 - DNA, 60 bp, Synthetic construct
  source 1..60: mol_type = other DNA
gagagcgaggtggcttcaataagcctggtggtcctccccttggaggggcacaaacgatca

SEQ ID NO: 88 - DNA, 94 bp, Synthetic construct
  source 1..94: mol_type = other DNA
tggtacagaggtaaatgcatgcgtagagttcagcagccttatagaccagcctgtttaccctgatgtgctggcatcgggaaggtgggggagctca

SEQ ID NO: 89 - DNA, 66 bp, Synthetic construct
  source 1..66: mol_type = other DNA
gacccatggatgaaggaccagatcttgatctaggttcactgctggcctataatacaacctcccaca

SEQ ID NO: 90 - DNA, 62 bp, Synthetic construct
  source 1..62: mol_type = other DNA
tagccaacagagcagcagctacgggcagcagaatcccgtcggagacggtctcttcaaggacg

SEQ ID NO: 91 - DNA, 48 bp, Synthetic construct
  source 1..48: mol_type = other DNA
cagagcagcagctacgggcagcagaggggtgcagcttttattttccca

SEQ ID NO: 92 - DNA, 59 bp, Synthetic construct
  source 1..59: mol_type = other DNA
gccaacagagcagcagctacgggcagcagaatttaccatatgagccccccaggagatca

SEQ ID NO: 93 - DNA, 60 bp, Synthetic construct
  source 1..60: mol_type = other DNA
ccatggatgaaggaccagatcttgatctagatttaccatatgagccccccaggagatcag

SEQ ID NO: 94 - DNA, 59 bp, Synthetic construct
  source 1..59: mol_type = other DNA
gccaacagagcagcagctacgggcagcagatttaccatatgagccccccaggagatcag

SEQ ID NO: 95 - DNA, 71 bp, Synthetic construct
  source 1..71: mol_type = other DNA
atatagccaacagagcagcagctacgggcagcagaatttaccatatgagccccccaggagatcagcctgga

SEQ ID NO: 96 - DNA, 45 bp, Synthetic construct
  source 1..45: mol_type = other DNA
ggaggacgcggtggaatgggaaaaattttgaaagacttatcttct

SEQ ID NO: 97 - DNA, 60 bp, Synthetic construct
  source 1..60: mol_type = other DNA
tgggcggggaggaggacgcggtggaatgggaaaaattttgaaagacttatcttctgaaga

SEQ ID NO: 98 - DNA, 60 bp, Synthetic construct
  source 1..60: mol_type = other DNA
gccaacagagcagcagctacgggcagcagagtgagaaaccataccagtgtgacttcaagg

SEQ ID NO: 99 - DNA, 51 bp, Synthetic construct
  source 1..51: mol_type = other DNA
cagagcagcagctacgggcagcagagtgagaaaccataccagtgtgacttc

SEQ ID NO: 100 - DNA, 59 bp, Synthetic construct
  source 1..59: mol_type = other DNA
gccaacagagcagcagctacgggcagcagagtgagaaaccataccagtgtgacttcaag

SEQ ID NO: 101 - DNA, 48 bp, Synthetic construct
  source 1..48: mol_type = other DNA
agagcagcagctacgggcagcagagtgagaaaccataccagtgtgact

SEQ ID NO: 102 - DNA, 48 bp, Synthetic construct; identical to SEQ ID NO: 101
  source 1..48: mol_type = other DNA

SEQ ID NO: 103 - DNA, 58 bp, Synthetic construct
  source 1..58: mol_type = other DNA
gccaacagagcagcagctacgggcagcagagtgagaaaccataccagtgtgacttcaa

SEQ ID NO: 104 - DNA, 63 bp, Synthetic construct
  source 1..63: mol_type = other DNA
agccaacagagcagcagctacgggcagcaggtgagaaaccataccagtgtgacttcaaggact

SEQ ID NO: 105 - DNA, 60 bp, Synthetic construct; identical to SEQ ID NO: 98
  source 1..60: mol_type = other DNA

SEQ ID NO: 106 - DNA, 65 bp, Synthetic construct
  source 1..65: mol_type = other DNA
tatagccaacagagcagcagctacgggcagcagagtgagaaaccataccagtgtgacttcaagga

SEQ ID NO: 107 - DNA, 60 bp, Synthetic construct; identical to SEQ ID NO: 98
  source 1..60: mol_type = other DNA

SEQ ID NO: 108 - DNA, 51 bp, Synthetic construct
  source 1..51: mol_type = other DNA
cgaggtggcttcaataagcctggtggtgagaaaccataccagtgtgacttc

SEQ ID NO: 109 - DNA, 58 bp, Synthetic construct
  source 1..58: mol_type = other DNA
agagcgaggtggcttcaataagcctggtgtgagaaaccataccagtgtgacttcaagg

SEQ ID NO: 110 - DNA, 60 bp, Synthetic construct
  source 1..60: mol_type = other DNA
gagagcgaggtggcttcaataagcctggtggtgagaaaccataccagtgtgacttcaagg

SEQ ID NO: 111 - DNA, 89 bp, Synthetic construct
  source 1..89: mol_type = other DNA
aggacaagaagtggaaggcttagtagttctgagctctcacctctcacccccccgtcttctgtctcttcctcgttaagcatttctgttag

SEQ ID NO: 112 - DNA, 60 bp, Synthetic construct
  source 1..60: mol_type = other DNA
aacccggtcagcaacggcctgtctcctcagaattcaattcgtcataatctgtccctacac

SEQ ID NO: 113 - DNA, 60 bp, Synthetic construct
  source 1..60: mol_type = other DNA
ccccagtgatctggcctcagacaactactggcataagctggaagtcacaccagtagtagc

SEQ ID NO: 114 - DNA, 67 bp, Synthetic construct
  source 1..67: mol_type = other DNA
acggagcagccccctggagctgaggctccgagtccgtggtggggctggggccggggtccccaggtcg

SEQ ID NO: 115 - DNA, 67 bp, Synthetic construct
  source 1..67: mol_type = other DNA
agggggccctgaccccacctcacccagctcggactctccgtggtggggctggggccggggtccccag

SEQ ID NO: 116 - DNA, 60 bp, Synthetic construct
  source 1..60: mol_type = other DNA
tgaccccacctcacccagctcggactctgggcggaccgccgtcaccggatcccagaccgc

SEQ ID NO: 117 - DNA, 60 bp, Synthetic construct
  source 1..60: mol_type = other DNA
ccccagcagaggccttatggatatgaccagatcatgcccaagaagccagcagaggaagga

SEQ ID NO: 118 - DNA, 51 bp, Synthetic construct
  source 1..51: mol_type = other DNA
cgaacaaaagtcctagggcaatcagtctaaatgacatttattatgcttcgg

SEQ ID NO: 119 - DNA, 72 bp, Synthetic construct
  source 1..72: mol_type = other DNA
tgaattagctagtagggagaagacagaagataaagtaaaccccttatttataaaggtgaggggaagtggctt

SEQ ID NO: 120 - DNA, 60 bp, Synthetic construct
  source 1..60: mol_type = other DNA
aaactctcctttgggtacgtccgtccgtccgtccgtttgtgacccacgcggtgcatgacc

SEQ ID NO: 121 - protein, 9 aa, Synthetic construct
SYGQQNPSY

SEQ ID NO: 122 - protein, 9 aa, Synthetic construct
QQNPSYDSV

SEQ ID NO: 123 - protein, 10 aa, Synthetic construct
SSYGQQNPSY

SEQ ID NO: 124 - protein, 11 aa, Synthetic construct
SSSYGQQNPSY

SEQ ID NO: 125 - protein, 12 aa, Synthetic construct
QSSSYGQQNPSY

SEQ ID NO: 126 - protein, 8 aa, Synthetic construct
QQSSLLAY

SEQ ID NO: 127 - protein, 9 aa, Synthetic construct
SSYGQQSSL

SEQ ID NO: 128 - protein, 9 aa, Synthetic construct
YGQQSSLLA

SEQ ID NO: 129 - protein, 9 aa, Synthetic construct
SYGQQSSLL

SEQ ID NO: 130 - protein, 9 aa, Synthetic construct
GQQSSLLAY

SEQ ID NO: 131 - protein, 10 aa, Synthetic construct
SYGQQSSLLA

SEQ ID NO: 132 - protein, 10 aa, Synthetic construct
YGQQSSLLAY

SEQ ID NO: 133 - protein, 11 aa, Synthetic construct
SSSYGQQSSLL

SEQ ID NO: 134 - protein, 11 aa, Synthetic construct
SSYGQQSSLLA

SEQ ID NO: 135 - protein, 11 aa, Synthetic construct
SYGQQSSLLAY

SEQ ID NO: 136 - protein, 12 aa, Synthetic construct
SSSYGQQSSLLA

SEQ ID NO: 137 - protein, 12 aa, Synthetic construct
SSYGQQSSLLAY

SEQ ID NO: 138 - protein, 13 aa, Synthetic construct
SSSYGQQSSLLAY

SEQ ID NO: 139 - protein, 9 aa, Synthetic construct
YGQQNPYQI

SEQ ID NO: 140 - protein, 9 aa, Synthetic construct
SSYGQQNPY

SEQ ID NO: 141 - protein, 9 aa, Synthetic construct
GQQNPYQIL

SEQ ID NO: 142 - protein, 10 aa, Synthetic construct
SSSYGQQNPY

SEQ ID NO: 143 - protein, 10 aa, Synthetic construct
SYGQQNPYQI

SEQ ID NO: 144 - protein, 11 aa, Synthetic construct
QSSSYGQQNPY

SEQ ID NO: 145 - protein, 11 aa, Synthetic construct
SYGQQNPYQIL

SEQ ID NO: 146 - protein, 9 aa, Synthetic construct
SYGQQNLPY

SEQ ID NO: 147 - protein, 10 aa, Synthetic construct
SSYGQQNLPY

SEQ ID NO: 148 - protein, 11 aa, Synthetic construct
SSSYGQQNLPY

SEQ ID NO: 149 - protein, 9 aa, Synthetic construct
QSSGQIQLW

SEQ ID NO: 150 - protein, 9 aa, Synthetic construct
QQSSGQIQL

SEQ ID NO: 151 - protein, 10 aa, Synthetic construct
GQQSSGQIQL

SEQ ID NO: 152 - protein, 10 aa, Synthetic construct
QQSSGQIQLW

SEQ ID NO: 153 - protein, 11 aa, Synthetic construct
YGQQSSGQIQL

SEQ ID NO: 154 - protein, 11 aa, Synthetic construct
GQQSSGQIQLW

SEQ ID NO: 155 - protein, 11 aa, Synthetic construct
QSSGQIQLWQF

SEQ ID NO: 156 - protein, 9 aa, Synthetic construct
KPGAPLPHL

SEQ ID NO: 157 - protein, 9 aa, Synthetic construct
FGGSGQIQL

SEQ ID NO: 158 - protein, 9 aa, Synthetic construct
GGSGQIQLW

SEQ ID NO: 159 - protein, 10 aa, Synthetic construct
FGGSGQIQLW

SEQ ID NO: 160 - protein, 11 aa, Synthetic construct
KFGGSGQIQLW

SEQ ID NO: 161 - protein, 9 aa, Synthetic construct
SSYGQQSEK

SEQ ID NO: 162 - protein, 9 aa, Synthetic construct
QQSEKPYQC

SEQ ID NO: 163 - protein, 10 aa, Synthetic construct
SSSYGQQSEK

SEQ ID NO: 164 - protein, 10 aa, Synthetic construct
SYGQQSEKPY

SEQ ID NO: 165 - protein, 11 aa, Synthetic construct
QSSSYGQQSEK

SEQ ID NO: 166 - protein, 11 aa, Synthetic construct
SSYGQQSEKPY

SEQ ID NO: 167 - protein, 11 aa, Synthetic construct
QQSEKPYQCDF

SEQ ID NO: 168 - protein, 12 aa, Synthetic construct
QQSSSYGQQSEK

SEQ ID NO: 169 - protein, 12 aa, Synthetic construct
SSSYGQQSEKPY

SEQ ID NO: 170 - protein, 13 aa, Synthetic construct
SQQSSSYGQQSEK

SEQ ID NO: 171 - DNA, 19 bp, Synthetic construct
  source 1..19: mol_type = other DNA
cagccagtcactgcacctc

SEQ ID NO: 172 - DNA, 19 bp, Synthetic construct
  source 1..19: mol_type = other DNA
gcttccagggttggctagg

SEQ ID NO: 173 - DNA, 20 bp, Synthetic construct
  source 1..20: mol_type = other DNA
tccggagaggagcagagata

SEQ ID NO: 174 - DNA, 19 bp, Synthetic construct
  source 1..19: mol_type = other DNA
atgctgcctgggtatagga

SEQ ID NO: 175 - DNA, 20 bp, Synthetic construct
  source 1..20: mol_type = other DNA
cgccctgcggtactataatc

SEQ ID NO: 176 - DNA, 20 bp, Synthetic construct
  source 1..20: mol_type = other DNA
tgcctcccactttctctttg

SEQ ID NO: 177 - DNA, 20 bp, Synthetic construct
  source 1..20: mol_type = other DNA
gcaagagaggcattctcacc

SEQ ID NO: 178 - DNA, 20 bp, Synthetic construct
  source 1..20: mol_type = other DNA
ggggtgttgaaggtctcaaa

SEQ ID NO: 179 - protein, 8 aa, Synthetic construct
SSYGQQNP

SEQ ID NO: 180 - protein, 8 aa, Synthetic construct
YGQQNPSY

SEQ ID NO: 181 - protein, 9 aa, Synthetic construct
SYGQQNPSY

SEQ ID NO: 182 - protein, 9 aa, Synthetic construct
QQNPSYDSV

SEQ ID NO: 183 - protein, 9 aa, Synthetic construct
SSYGQQNPS

SEQ ID NO: 184 - protein, 9 aa, Synthetic construct
QSSSYGQQN

SEQ ID NO: 185 - protein, 9 aa, Synthetic construct
SSSYGQQNP

SEQ ID NO: 186 - protein, 9 aa, Synthetic construct
YGQQNPSYD

SEQ ID NO: 187 - protein, 9 aa, Synthetic construct
GQQNPSYDS

SEQ ID NO: 188 - protein, 9 aa, Synthetic construct
QNPSYDSVR

SEQ ID NO: 189 - protein, 10 aa, Synthetic construct
SSYGQQNPSY

SEQ ID NO: 190 - protein, 10 aa, Synthetic construct
GQQNPSYDSV

SEQ ID NO: 191 - protein, 10 aa, Synthetic construct
QQNPSYDSVR

SEQ ID NO: 192 - protein, 10 aa, Synthetic construct
QQSSSYGQQN

SEQ ID NO: 193 - protein, 10 aa, Synthetic construct
QSSSYGQQNP

SEQ ID NO: 194 - protein, 10 aa, Synthetic construct
SSSYGQQNPS

SEQ ID NO: 195 - protein, 10 aa, Synthetic construct
SYGQQNPSYD

SEQ ID NO: 196 - protein, 10 aa, Synthetic construct
YGQQNPSYDS

SEQ ID NO: 197 - protein, 10 aa, Synthetic construct
QNPSYDSVRR

SEQ ID NO: 198 - protein, 11 aa, Synthetic construct
SSSYGQQNPSY

SEQ ID NO: 199 - protein, 11 aa, Synthetic construct
QQNPSYDSVRR

SEQ ID NO: 200 - protein, 11 aa, Synthetic construct
GQQNPSYDSVR

SEQ ID NO: 201 - protein, 11 aa, Synthetic construct
YGQQNPSYDSV

SEQ ID NO: 202 - protein, 11 aa, Synthetic construct
SSYGQQNPSYD

SEQ ID NO: 203 - protein, 11 aa, Synthetic construct
SYGQQNPSYDS

SEQ ID NO: 204 - protein, 12 aa, Synthetic construct
QSSSYGQQNPSY

SEQ ID NO: 205 - protein, 12 aa, Synthetic construct
SSSYGQQNPSYD

SEQ ID NO: 206 - protein, 12 aa, Synthetic construct
SSYGQQNPSYDS

SEQ ID NO: 207 - protein, 12 aa, Synthetic construct
SYGQQNPSYDSV

SEQ ID NO: 208 - protein, 12 aa, Synthetic construct
GQQNPSYDSVRR

SEQ ID NO: 209 - protein, 13 aa, Synthetic construct
QQSSSYGQQNPSY

SEQ ID NO: 210 - protein, 13 aa, Synthetic construct
SSYGQQNPSYDSV

SEQ ID NO: 211 - protein, 13 aa, Synthetic construct
SYGQQNPSYDSVR

SEQ ID NO: 212 - protein, 14 aa, Synthetic construct
SQQSSSYGQQNPSY

SEQ ID NO: 213 - protein, 8 aa, Synthetic construct
SSYGQQSS

SEQ ID NO: 214 - protein, 8 aa, Synthetic construct
SYGQQSSL

SEQ ID NO: 215 - protein, 8 aa, Synthetic construct
YGQQSSLL

SEQ ID NO: 216 - protein, 8 aa, Synthetic construct
QQSSLLAY

SEQ ID NO: 217 - protein, 9 aa, Synthetic construct
SSYGQQSSL

SEQ ID NO: 218 - protein, 9 aa, Synthetic construct
YGQQSSLLA

SEQ ID NO: 219 - protein, 9 aa, Synthetic construct
SYGQQSSLL

SEQ ID NO: 220 - protein, 9 aa, Synthetic construct
GQQSSLLAY

SEQ ID NO: 221 - protein, 9 aa, Synthetic construct
SSSYGQQSS

SEQ ID NO: 222 - protein, 9 aa, Synthetic construct
QSSSYGQQS

SEQ ID NO: 223 - protein, 10 aa, Synthetic construct
SYGQQSSLLA

SEQ ID NO: 224 - protein, 10 aa, Synthetic construct
YGQQSSLLAY

SEQ ID NO: 225 - protein, 10 aa, Synthetic construct
SSSYGQQSSL

SEQ ID NO: 226 - protein, 10 aa, Synthetic construct
SSYGQQSSLL

SEQ ID NO: 227 - protein, 10 aa, Synthetic construct
GQQSSLLAYN

SEQ ID NO: 228 - protein, 11 aa, Synthetic construct
SSSYGQQSSLL

SEQ ID NO: 229 - protein, 11 aa, Synthetic construct
SSYGQQSSLLA

SEQ ID NO: 230 - protein, 11 aa, Synthetic construct
SYGQQSSLLAY

SEQ ID NO: 231 - protein, 11 aa, Synthetic construct
QSSSYGQQSSL

SEQ ID NO: 232 - protein, 11 aa, Synthetic construct
YGQQSSLLAYN

SEQ ID NO: 233 - protein, 12 aa, Synthetic construct
QQSSSYGQQSSL

SEQ ID NO: 234 - protein, 12 aa, Synthetic construct
SSSYGQQSSLLA

SEQ ID NO: 235 - protein, 12 aa, Synthetic construct
SSYGQQSSLLAY

SEQ ID NO: 236 - protein, 13 aa, Synthetic construct
SSSYGQQSSLLAY

SEQ ID NO: 237 - protein, 9 aa, Synthetic construct
SSYGQQSEK

SEQ ID NO: 238 - protein, 9 aa, Synthetic construct
QQSEKPYQC

SEQ ID NO: 239 - protein, 10 aa, Synthetic construct
SSSYGQQSEK

SEQ ID NO: 240 - protein, 10 aa, Synthetic construct
SYGQQSEKPY

SEQ ID NO: 241 - protein, 11 aa, Synthetic construct
QSSSYGQQSEK

SEQ ID NO: 242 - protein, 11 aa, Synthetic construct
SSYGQQSEKPY

SEQ ID NO: 243 - protein, 11 aa, Synthetic construct
QQSEKPYQCDF

SEQ ID NO: 244 - protein, 12 aa, Synthetic construct
QQSSSYGQQSEK

SEQ ID NO: 245 - protein, 12 aa, Synthetic construct
SSSYGQQSEKPY

SEQ ID NO: 246 - protein, 13 aa, Synthetic construct
SQQSSSYGQQSEK

(19) United States
(12) Patent Application Publication
(10) Pub. No.: US 2026/0250358 A1
(43) Pub. Date: Aug. 27, 2026

(54) FUSION NEOANTIGENS FOR USE IN CANCER VACCINES, ANTIBODY THERAPY, AND CELL THERAPIES

(71) Applicant: THE CLEVELAND CLINIC FOUNDATION, Cleveland, OH (US)

(72) Inventors: Timothy A. CHAN, Cleveland, OH (US); Jeffrey KNAUF, Cleveland, OH (US); Suheyla HASGUR, Cleveland, OH (US); Gyanu PARAJULI, Cleveland, OH (US)

(21) Appl. No.: 19/127,884

(22) PCT Filed: Nov. 13, 2023
(86) PCT No.: PCT/US2023/079503

§ 371 Date: May 7, 2025

Related U.S. Application Data
(60) Provisional application No. 63/384,027, filed on Nov. 16, 2022.
(60) Provisional application No. 63/384,158, filed on Nov. 17, 2022.

(51) Int. Cl. C07K 14/82; A61K 38/00; A61K 40/11; A61K 40/31; A61K 40/42; A61K 45/06; C07K 14/47; C07K 16/28; C07K 16/32; C12N 15/85; C12N 15/88; C12Q 1/6881

(52) U.S. Cl. CPC C07K 14/82; A61K 40/11; A61K 40/31; A61K 40/4201; A61K 45/06; C07K 14/4702; C07K 16/2833; C07K 16/32; C12N 15/85; C12N 15/88; C12Q 1/6881; A61K 38/00; C07K 2317/32; C07K 2319/00; C12N 2800/22; C12Q 2600/106

(57) ABSTRACT

Provided herein are compositions, systems, kits, and methods for treating a patient with cancer (e.g., Ewing's Sarcoma) by treating the patient with at least one of the following compositions: a) a composition comprising at least a portion of at least one fusion neoantigen selected from: EWSR1-FLi1, EWS-FEV, EWSR1-ERG, EWS-ATF, EWS-WT1, PAX7-FOXO1, CCNB3-BCOR, CIC-DVX, SS18-SSX2; b) a composition comprising a nucleic acid sequence encoding said at least a portion of said at least one fusion neoantigen; c) a composition comprising an antibody, or antigen-binding fragment, the specifically binds said at least a portion of said at least one fusion neoantigen; and/or d) a composition comprising a chimeric antigen receptor T-cell (CAR T cell) that expresses a chimeric antigen receptor that is specific for said at least a portion of said at least one fusion neoantigen.

### CROSS-REFERENCE TO RELATED APPLICATIONS

This application is a 371 national stage application of PCT/US2023/079503, filed Nov. 13, 2023, which claims the benefit of U.S. Provisional Application Nos. 63/384,027, filed Nov. 16, 2022, and 63/384,158, filed Nov. 17, 2022, all of which are hereby incorporated by reference in their entirety.

### SEQUENCE LISTING

The instant application contains a Sequence Listing which has been submitted electronically in XML. format and is hereby incorporated by reference in its entirety. Said XML. copy created on Nov. 13, 2023, is named CCF_4149_601_Sequence Listing.xml and is 218,815 bytes.

### FIELD

Provided herein are compositions, systems, kits, and methods for treating a patient with cancer (e.g., Ewing's Sarcoma) by treating the patient with at least one of the following compositions: a) a composition comprising at least a portion of at least one fusion neoantigen selected from: EWSR1-FLi1, EWS-FEV, EWSR1-ERG, EWS-ATF, EWS-WT1, PAX7-FOXO1, CCNB3-BCOR, CIC-DVX, SS18-SSX2; b) a composition comprising a nucleic acid sequence encoding said at least a portion of said at least one fusion neoantigen; c) a composition comprising an antibody, or antigen-binding fragment, the specifically binds said at least a portion of said at least one fusion neoantigen; and/or d) a composition comprising a chimeric antigen receptor T-cell (CAR T cell) that expresses a chimeric antigen receptor that is specific for said at least a portion of said at least one fusion neoantigen.

### BACKGROUND

Ewing sarcoma (EWS) is the second most common and aggressive type of metastatic bone and soft tissue sarcoma with high mortality rates in pediatric patients and young adults. The survival of patients with metastatic EWS remains less than 20% by the frequent occurrence of incurable systemic relapses. Given the dismal long-term outcomes and very intensive therapies including surgery or radiation of the primary tumor site, and multiagent chemotherapy used, there is an urgent need to explore and develop alternative treatment modalities including immunotherapies.

### SUMMARY

Provided herein are compositions, systems, kits, and methods for treating a patient with cancer (e.g., Ewing's Sarcoma) by treating the patient with at least one of the following compositions: a) a composition comprising at least a portion of at least one fusion neoantigen selected from: EWSR1-FLi1, EWS-FEV, EWSR1-ERG, EWS-ATF, EWS-WT1, PAX7-FOXO1, CCNB3-BCOR, CIC-DVX, SS18-SSX2; b) a composition comprising a nucleic acid sequence encoding said at least a portion of said at least one fusion neoantigen; c) a composition comprising an antibody, or antigen-binding fragment, the specifically binds said at least a portion of said at least one fusion neoantigen; and/or d) a composition comprising a chimeric antigen receptor T-cell (CAR T cell) that expresses a chimeric antigen receptor that is specific for said at least a portion of said at least one fusion neoantigen.

In certain embodiments, provided herein are compositions comprising: a) at least a portion of (e.g., 50% . . . 75% . . . or 100% of) at least one fusion neoantigen selected from: i) an EWSR1-FLi1 fusion protein, ii) an EWS-FEV fusion protein; iii) an EWSR1-ERG fusion protein, iv) an EWS-ATF fusion protein; v) an EWS-WT1 fusion protein; vi) a KMT2A c2318 dupC fusion protein; vii) a PAX7-FOXO1 fusion protein; viii) a CCNB3-BCOR fusion protein; ix) CIC-DVX fusion protein; x) SS18-SSX2 fusion protein; xi) a FN1fs p.F1778 fusion protein; xii) 1 ATRX pD20GG fusion protein; and xiii) frameshift 2:2FHX3 p.F297 fusion protein, wherein the at least one fusion neoantigen is purified and isolated; b) a nucleic acid vector comprising a nucleic acid sequence encoding at least a portion of the at least one fusion neoantigen; c) an isolated and purified antibody, or antigen-binding fragment of the antibody, the specifically binds at least a portion of the at least one fusion neoantigen; d) an antigen presenting cell (APC) that comprises at least a portion of the at least one fusion neoantigen, and/or e) a chimeric antigen receptor T-cell (CAR T cell) that expresses a chimeric antigen receptor that is specific for at least a portion of the at least one fusion neoantigen. In some embodiments, at least one of the following applies: i) wherein said polynucleotide comprises RNA, and optionally wherein said RNA is partially or fully human codon optimized; ii) wherein said polynucleotide further comprises or encodes: a 5′ untranslated region (UTR), a 5′ cap, a 3′ UTR, a 3′ tailing sequence, or any combination thereof; iii) wherein the polynucleotide comprises at least one chemically modified nucleotide, which is optionally a modified uracil; iv) wherein at least 60% of the uracil in the polynucleotide encoding are chemically modified; v) wherein the at least one chemically modified nucleotide comprises 5-methylcytosine or N1-methylpseudouridine (m1Ψ); vi) wherein the composition further comprises a delivery vehicle, and optionally wherein the said delivery vehicle comprises a lipid nanoparticle encapsulating the polynucleotide, and further optionally wherein the lipid nanoparticle comprises a cationic lipid, a neutral and/or non-cationic lipid, a sterol, or any combination thereof, and further optionally wherein the non-cationic lipid comprises a phospholipid and/or wherein the sterol comprises cholesterol or a modification or ester thereof and/or wherein the lipid nanoparticle comprises a polyethylene glycol (PEG)-lipid conjugate.

In some embodiments, provided herein are methods of treating a patient with cancer comprising: treating a patient that has cancer with a composition comprising: a) at least a portion of at least one fusion neoantigen selected from: i) an EWSR1-FLi1 fusion protein, ii) an EWS-FEV fusion protein; iii) an EWSR1-ERG fusion protein, iv) an EWS-ATF fusion protein; v) an EWS-WT1 fusion protein; vi) a KMT2A c2318 dupC fusion protein; vii) a PAX7-FOXO1 fusion protein; viii) a CCNB3-BCOR fusion protein; ix) CIC-DVX fusion protein; x) SS18-SSX2 fusion protein; xi) a FN1fs p.F1778 fusion protein; xii) 1 ATRX pD20GG fusion protein; and xiii) frameshift 2:2FHX3 p.F297 fusion protein; b) a nucleic acid sequence encoding the at least a portion of the at least one fusion neoantigen; c) an antibody, or antigen-binding fragment of the antibody, the specifically binds the at least a portion of the at least one fusion neoantigen; and/or d) a chimeric antigen receptor T-cell (CAR T cell) that expresses a chimeric antigen receptor that is specific for the at least a portion of the at least one fusion neoantigen.

In some embodiments, the methods further comprise: testing a sample (e.g., blood sample or tissue sample) from said patient to determine at least one HLA type for said patient, and/or receiving results of testing said sample for said at least one HLA type of said patient, and wherein said at least a portion of said at least one fusion neoantigen has an HLA type that is compatible with, or matches type, or type and subtype, of said at least one HLA type of said patient. In matching HLA type of the fusion antigen employed and the patient's HLA type one can consult, FIGS. 1, Table 1, and FIG. 8 (e.g., after performing an HLA typing assay on a sample from said patient, such as using a commercial next generation sequencing HLA typing service).

In particular embodiments, the at least one fusion neoantigen is selected from any of SEQ ID NOs: 1-51, 121-170, and 179-246, and/or wherein the nucleic acid sequence encoding the at least a portion of the at least one fusion neoantigen is selected from SEQ ID NOs: 52-120. In other embodiments, the at least a portion of the at least one fusion neoantigen comprises: i) at least a portion of the EWSR1 and the FLi1 proteins; ii) at least a portion of the EWSR1 and the ERG proteins; iii) at least a portion of the FUS and the ERG proteins; or the EWSR1 and the WT1 proteins.

In certain embodiments, wherein: i) the EWS-FLi1 fusion protein is an exon 7-exon 5, 6, 7, 8, or 10 exon fusion protein; ii) the EWS-FLi1 fusion protein is an exon 10-exon 5, 6, or 7 exon fusion protein; iii) the EWS-FLi1 fusion protein is an exon 9-exon 8 fusion protein; iv) the EWS-FLi1 fusion protein is an intron 10-intron 4 fusion protein; v) the EWS-FEW fusion protein is an exon 7-exon 2 fusion protein; vi) the EWS-ERG fusion protein is an exon 7-exon 8 or 9 fusion protein; vii) the EWS-ERG fusion protein is an exon 10-exon 9 fusion protein; viii) the EWS-ERG fusion protein is an exon 9-exon 7 fusion protein; ix) the EWS-ATF fusion protein is an exon 8-exon 4 fusion protein; x) the EWS-WT1 fusion protein is an exon 7-exon 7 or 8 fusion protein; xi) the EWS-WT1 fusion protein is an exon 9-exon 7 fusion protein; xii) the CIC-DVX fusion protein is an exon 20-exon 1 fusion protein; and/or xiii) the SS18-SSX2 fusion protein is an exon 10-exon 6 fusion protein.

In some embodiments, wherein the antibody, and the antigen-binding fragment of the antibody, specifically bind the at least a portion of the at least one fusion neoantigen when it is in a complex with a major histocompatibility complex molecule. In certain embodiments, the CAR T-cell specifically binds the at least a portion of the at least one fusion neoantigen when it is in a complex with a major histocompatibility complex molecule.

In additional embodiments, the methods further comprise determining if a sample collected from the patient is positive or negative for the at least one fusion neoantigen or nucleic acid encoding the at least one fusion neoantigen. In additional embodiments, the methods further comprise determining comprises performing an immuno-assay on the sample, or derivative thereof, to determine the presence or absence of the at least one fusion neoantigen. In some embodiments, the determining comprises performing a nucleic acid assay on the sample, or a derivative thereof, to determine the presence or absence of the at least one fusion neoantigen. In additional embodiments, the nucleic acid assay comprises sequencing DNA and/or RNA from the sample or the derivative thereof.

In certain embodiments, the composition further comprises an expression vector, and wherein the nucleic acid sequence is part of the expression vector. In other embodiments, the cancer comprises Ewing's sarcoma, or Alveolar Rhabdomyosarcoma, or Paraganglioma or Synovial sarcoma.

In particular embodiments, the methods further comprise treating the patient with an immune-checkpoint inhibitor. In other embodiments, the antibody, or antigen binding portion thereof, is monoclonal. In some embodiments, the antigen binding portion of the antibody is selected from: F(ab′)2, Fab, Fab′ and Fv.

In some embodiments, provided herein are systems or kits comprising: a) a composition as described above or otherwise herein, and b) a syringe vial and/or a syringe. In certain embodiments, the composition is located inside said syringe vial and/or said syringe.

### DESCRIPTION OF THE FIGURES

FIG. 1A-1T show various amino acid sequences (SEQ ID NOs: 1-51) and nucleic acid sequences (SEQ ID NOs: 52-120), that can be or encode the fusion neoantigens herein. Whole exome sequencing and RNAseq were used to identify fusion oncoprotein and HLA haplotype, respectively in sarcomas. A-R) Fusion identified in Ewing's sarcoma patients and the patient haplotype. S,T) Fusion identify in other sarcomas and the patients HLA haplotype. The mRNA and protein sequence flank the fusion junction. Blue sequence of the 5′ or n-terminal fusion partner of the fusion mRNA or protein respectively. Red the sequence of the 3′ or n-terminal partner. This figure shows the distribution and frequency of different fusion oncoproteins in Ewings sarcomas. Additionally, it demonstrates the HLA haplotype frequency in patients with Ewing's sarcoma harboring a given fusion oncoprotein.

FIG. 2: Screening of EWS fusions-derived peptides by cell-based peptide stabilization and immunogenicity with HLA-A*01:01⁺ cells. K562 cells were stably transfected with pCDNA3.1 plasmid expressing HLA-A*01:01. A) Representative flow cytometry of cells stained with FITC conjugated anti-B2M antibody (2M2) confirming expression of HLA-A*01:01 and HLA stabilization by addition of a neoantigen peptides from the type 2 EWSR1-FLI1 fusion. B, C) HLA stabilization assay in K562/HLA-A*01:01 cells incubated with neoantigen peptides following overnight incubation from the indicated EWS fusion proteins. Bars represent normalized fold change in the MFI of viable cells stained with FITC conjugated anti-B2M antibody. D) Healthy donor HLA-A*01:01⁺ antigen presenting cells (APCs) were pulsed with indicated peptides irradiated with 50Gy and then co-cultured with healthy donor CD8⁺ T cells. After 21-28 days, CD8⁺ T cells were subjected to IFN-γ ELISpot assay as directed by the manufacturer. Red boxed round peptides identified by mass spectrometry. This figure identifies peptides from EWRS1/FLI1 fusion that bind to HLA-A*01:01. Binding is further supported for the highest binders by demonstrating the peptide can promote T cell activation when presented by HLA-A*01:01. Red boxes indicate the peptides that were identified to be presented by HLA-A*0101 by mass spec. Peptides identified by mass spec, found to bind in HLA stabilization assay, and found to activate T cells represent very high confidence peptides for presentation by HLA-A*01:01.

FIG. 3: Screening of EWS fusions-derived peptides by cell-based peptide stabilization andimmunogenicity with HLA-A*30:02⁺ cells. K562 cells were stably transfected with pCDNA3.1 plasmid expressing HLA-A*30:02. A, B, C) HLA stabilization assay in K562/HLA-A*30:02 cells incubated with neoantigen peptides following overnight incubation from the indicated EWS fusion proteins. Bars represent normalized fold change in the MFI of viable cells stained with FITC conjugated anti-B2M antibody. D) Healthy donor HLA-A*30:02⁺ APCs were pulsed with indicated peptides irradiated with 50Gy, and then co-cultured with healthy donor CD8⁺ T cells. After 21-28 days, CD8⁺ T cells were subjected to IFN-γ ELISpot assay as directed by the manufacturer. This figure identifies peptides from EWRS1/FLI1 fusion that bind to HLA-A*30:02. Binding with further supported for the highest binders by demonstrating the peptide can promote T cell activation when presented by HLA-A*30:02. Peptides identified found to bind in HLA stabilization assay and found to activate T cells represent high confidence peptides for presentation by HLA-A*30:02.

FIG. 4: Screening of EWS fusions-derived peptides by cell-based peptide stabilization with HLA-A*24:02+ cells. K562 cells were stably transfected with pCDNA3.1 plasmid expressing HLA-A*24:02. A) Representative flow cytometry of cells stained with FITC conjugated anti-B2M antibody (2M2) confirming expression of HLA-A*24:02. B, C) HLA stabilization assay in K562/HLA-A*24:02 cells incubated with neoantigen peptides following overnight incubation from the indicated EWS fusion proteins. Red-boxed peptides are those identified by mass spectrometry. Bars represent normalized fold change in the MFI of viable cells stained with FITC conjugated anti-B2M antibody. This figure identifies peptides from EWRS1/FLI1 fusion that bind to HLA-A*24:02. Peptides found to bind the HLA in a HLA stabilization assay and found to activate T cells represent likely peptides for presentation by HLA-A*2402.

FIG. 5: Screening of EWS fusions derived peptides by cell-based peptide stabilization. K562 cells were stably transfected with pCDNA3.1 plasmid expressing HLA-A*02:05. A, B) HLA stabilization assay in K562/HLA-A*02:05 cells incubated with neoantigen peptides from EWSR1/FLI1 type 1 (A) or type 2 (B) fusion oncoprotein, following overnight incubation from the indicated EWS fusion proteins. Red boxed peptides are those identified by mass spec. Bars represent normalized fold change in the MFI of viable cells stained with FITC conjugated anti-B2M antibody. This figure identifies peptides from EWRS1/FLI1 fusion that bind to HLA-A*02:05 or A24:02. Peptides found to bind in HLA stabilization assay and found to activate T cells represent likely peptides for presentation by HLA-A*24:02.

FIG. 6: Validation of COS7 and the EWS cell line for LC MS/MS analysis. A-C) COS7 cells transfected with indicated HLAs and EWSR1-FLI1 fusion plasmids. A) Flow cytometry using antibodies to anti-FLI1 FITC, anti-HLA-ABC PE and anti-B2M-APC confirming expression of EWSR1-FLI1 fusion (left) and HLA-A*01:01 (right). B) Western blotting using antibodies to anti-FLI1 and anti-HLA-ABC. C) Quantitative RT-PCR with primers specific to the human HLA-A*0101. Bars represent β-actin normalized expression of HLA-A*01:01. D) Flow cytometry using PE conjugated anti-HLA-ABC of EWS cell line, A673. E) Agarose gel of RT-PCR product produced with primers specific to EWSR1-FLI1 fusion shows expected size band. F) Sanger sequence of RT-PCR product in (E) confirms EWSR1-FLI1 type 1 fusion in A673 cells.

FIG. 7: Validation of COS7 expressing EWSR1-FLI1 type 2 fusion for LC MS/MS analysis. A-D) Flow cytometry of transfected COS7 stained with anti-FLI1 FITC, anti-HLA-ABC PE and anti-B2M APC showing expression of EWSR1-FLI1 type 2 fusion (left) and indicated HLA (right) in EWSR1-FLI1+ viable cells. E) Flow cytometry of COS7 transfected with EWSR1-FLI1 type 2 fusion and the indicated HLA-A*02:01 stained with anti-HLA A02 confirming expression of HLA-A*02:01 in EWSR1-FLI1+ viable cells.

FIG. 8 shows neopeptides (SEQ ID NOs: 179-246) and the corresponding HLAs that were shown to present the respective peptide. Each peptide is presented in a row and the matching HLAs are listed.

### DETAILED DESCRIPTION

Provided herein are compositions, systems, kits, and methods for treating a patient with cancer (e.g., Ewing's Sarcoma) by treating the patient with at least one of the following compositions: a) a composition comprising at least a portion of at least one fusion neoantigen selected from: EWSR1-FLi1, EWS-FEV, EWSR1-ERG, EWS-ATF, EWS-WT1, PAX7-FOXO1, CCNB3-BCOR, CIC-DVX, SS18-SSX2; b) a composition comprising a nucleic acid sequence encoding said at least a portion of said at least one fusion neoantigen; c) a composition comprising an antibody, or antigen-binding fragment, the specifically binds said at least a portion of said at least one fusion neoantigen; and/or d) a composition comprising a chimeric antigen receptor T-cell (CAR T cell) that expresses a chimeric antigen receptor that is specific for said at least a portion of said at least one fusion neoantigen.

### I. Fusion Neoantigens

Neoantigens are cancer mutations that give rise to epitopes that can be targeted by the immune system. We have discovered that fusion neoantigens, or neoantigens that are formed by gene fusion events, can be excellent targets for immunotherapies such as cancer vaccines. We have previously shown that fusion neoantigens can be targeted by T cells and drive the rejection of tumors (Yang et al., Nature Medicine, 25:767-775, 2019). We have undertaken a large analysis of cancer genomes and shown that Ewing's Sarcoma and several other pediatric cancers have candidate immunogenic targets.

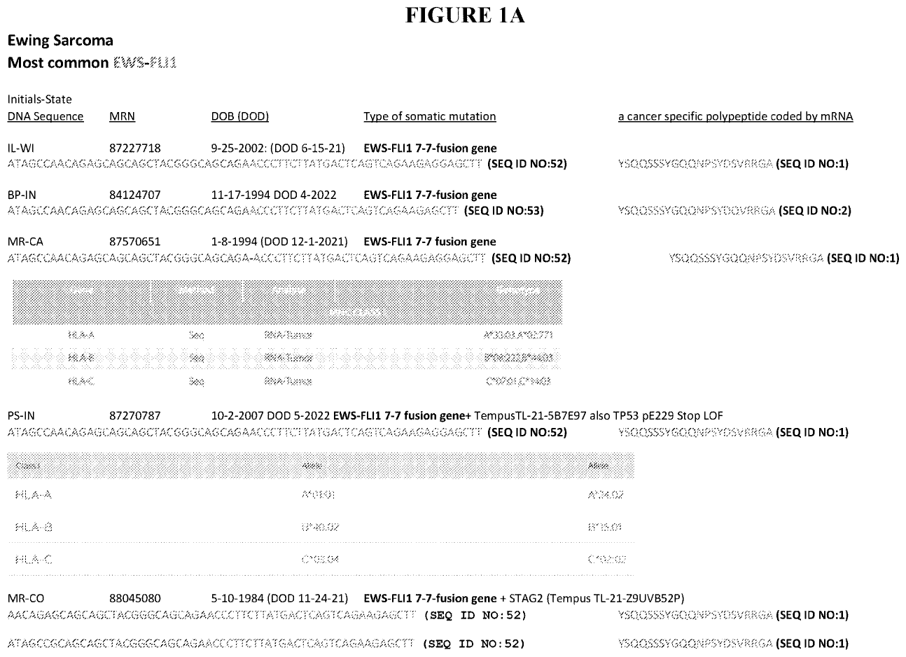

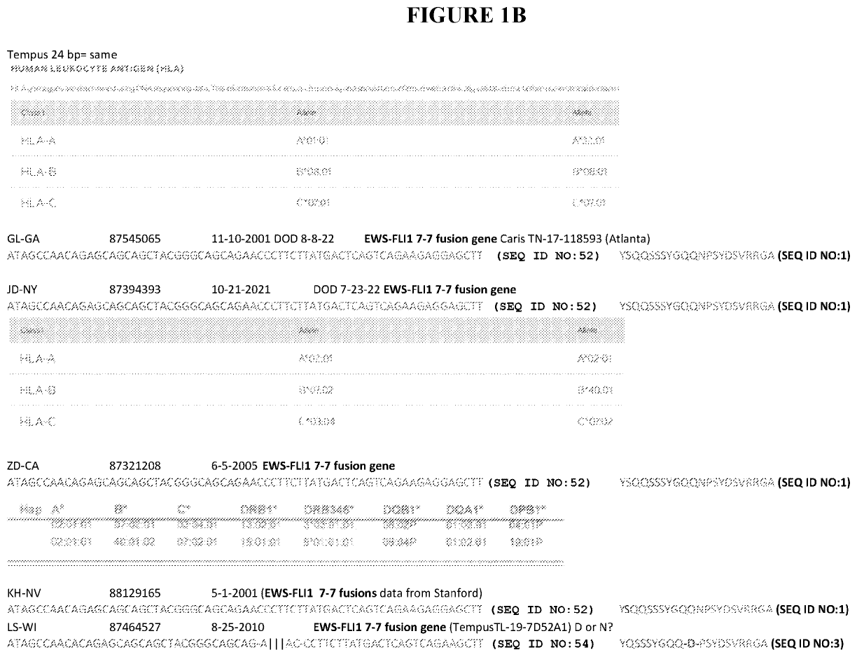

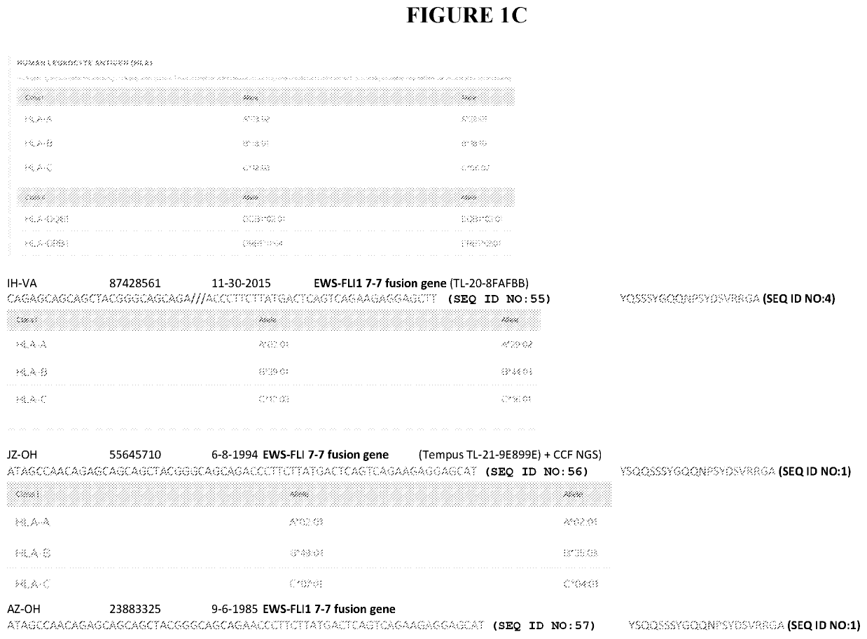

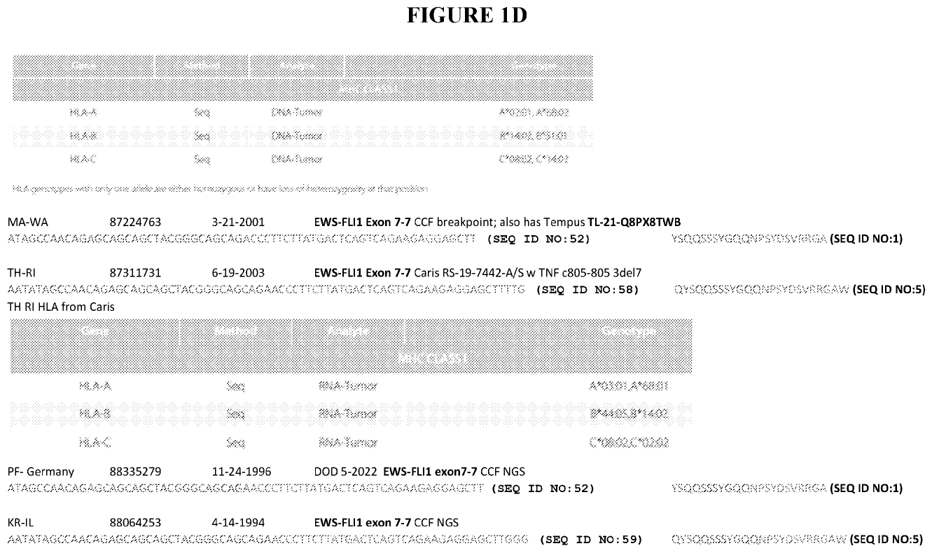

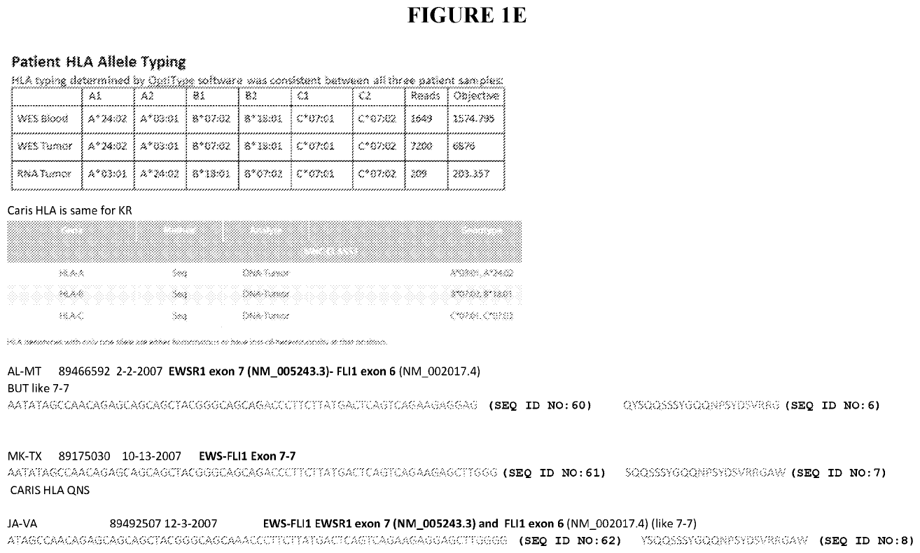

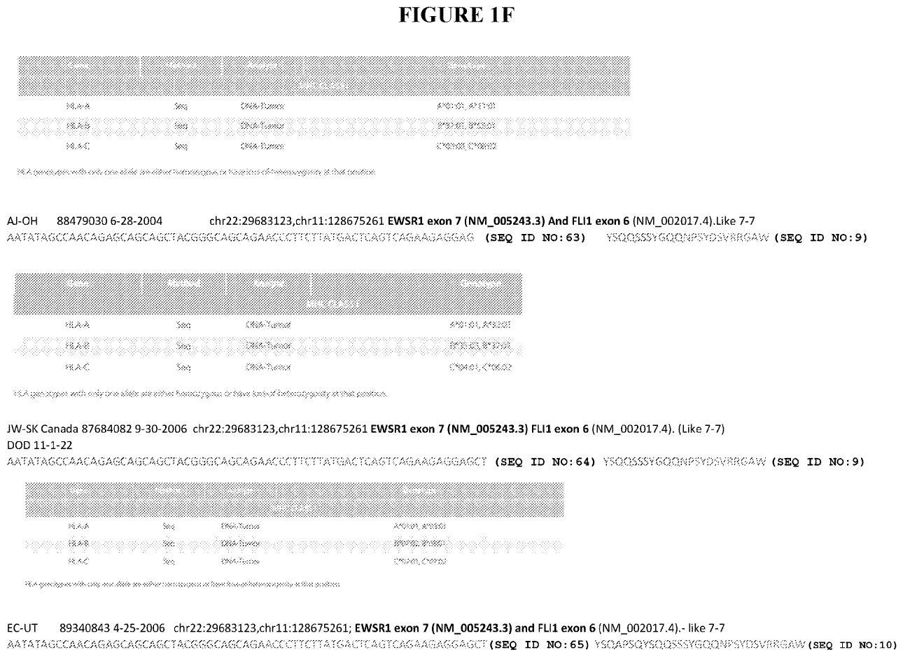

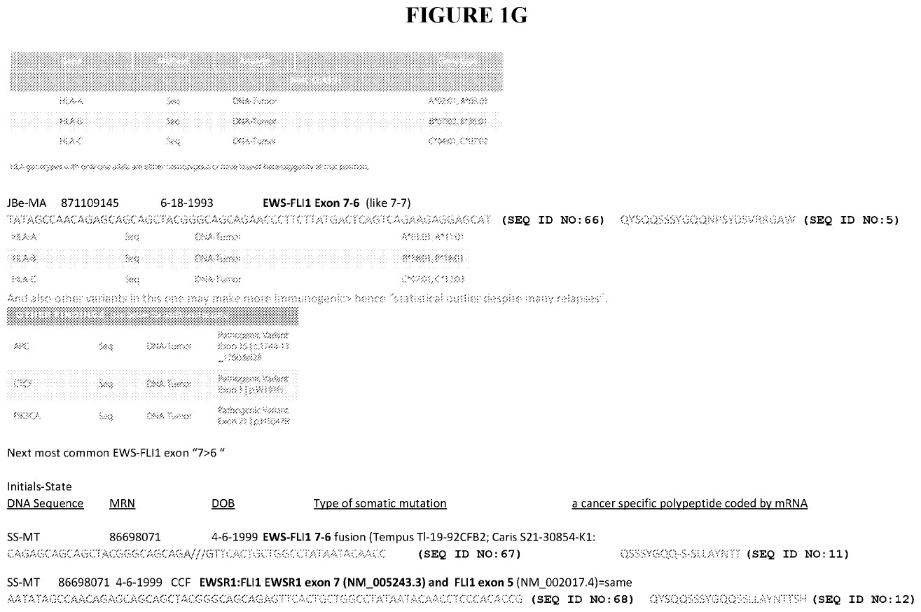

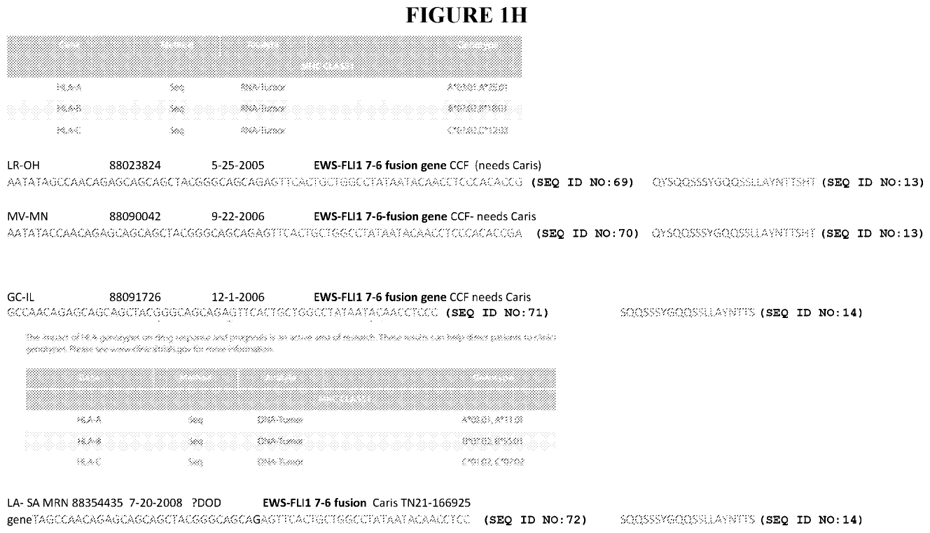

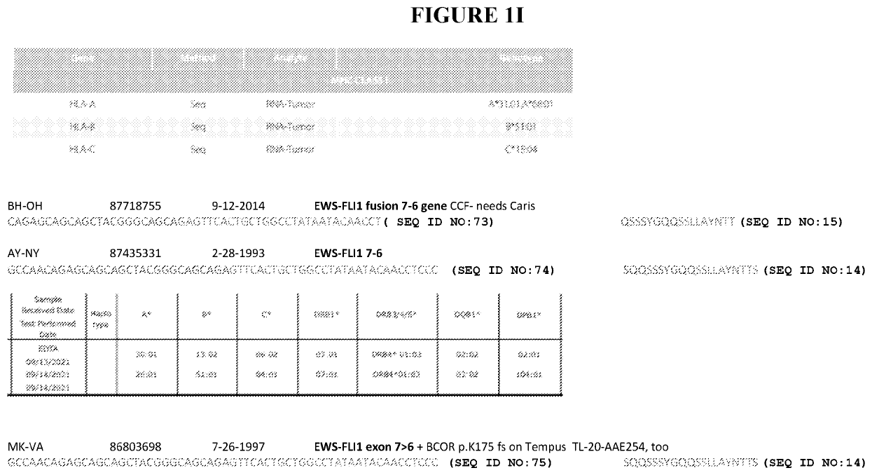

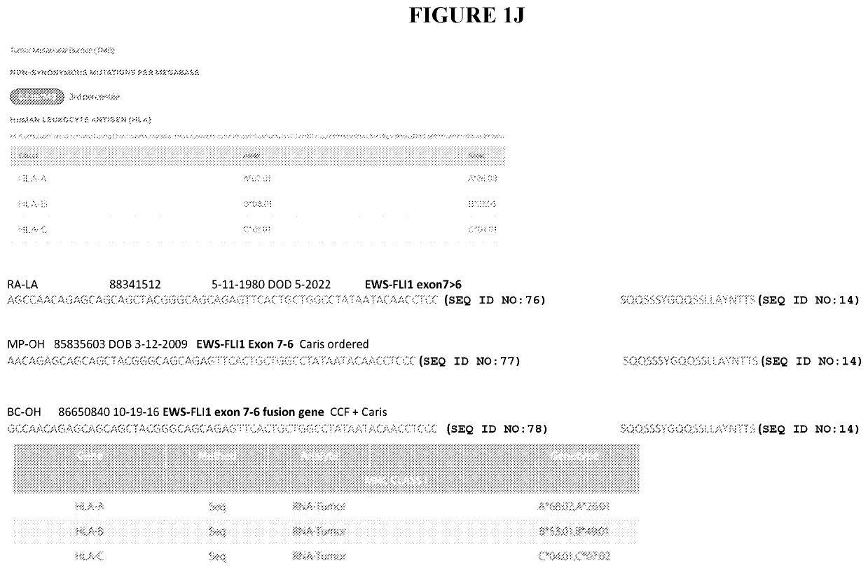

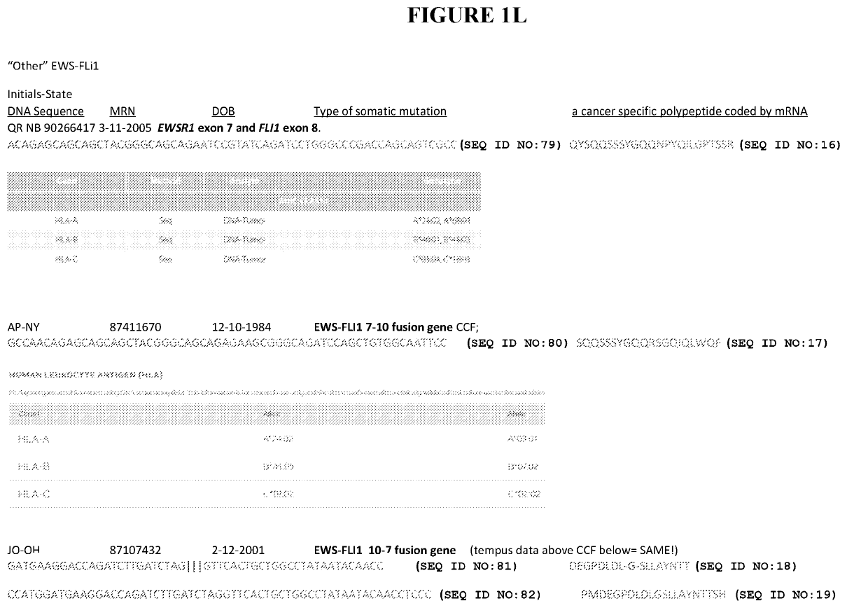

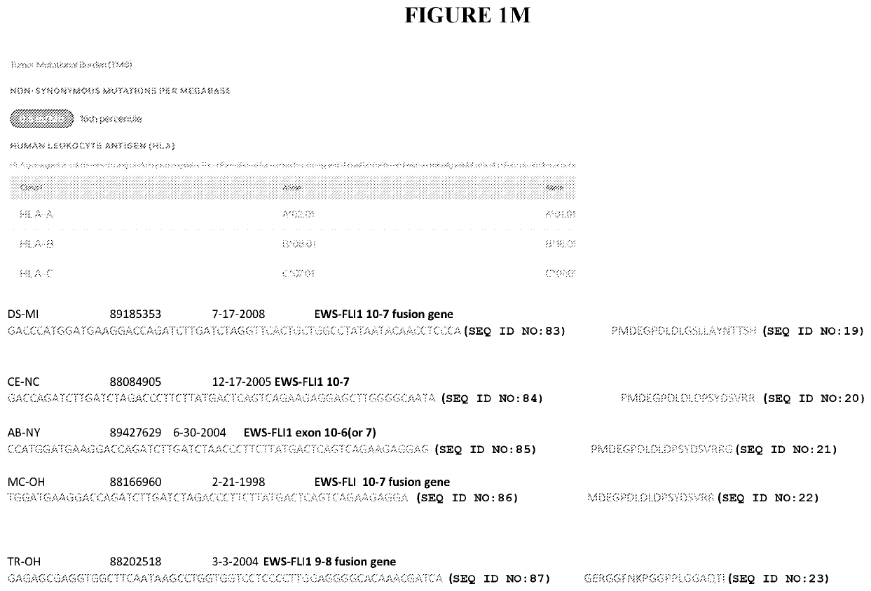

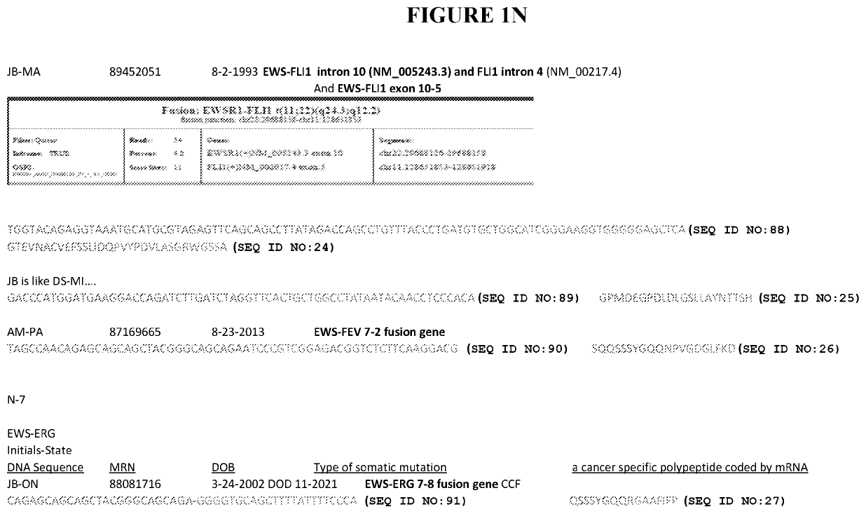

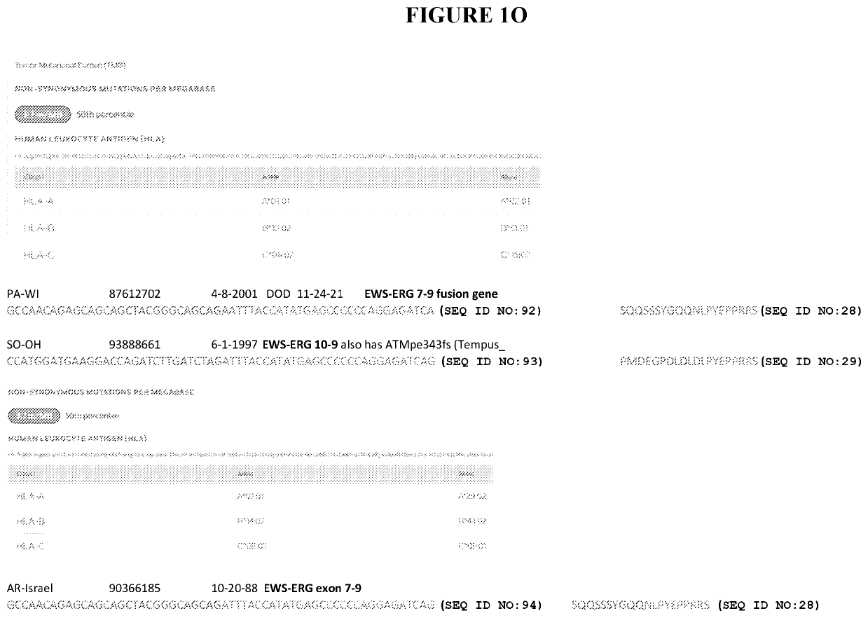

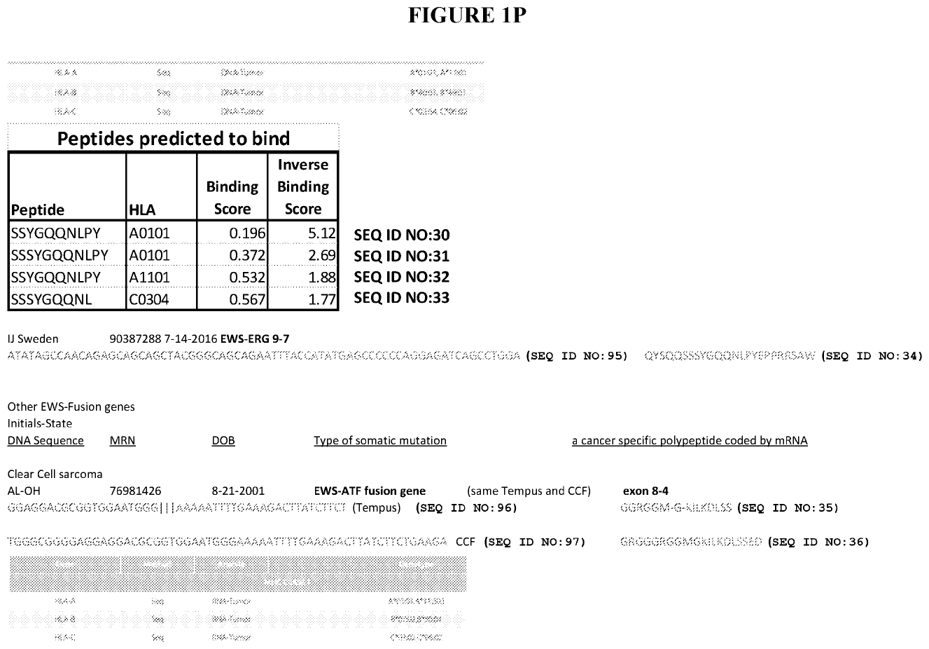

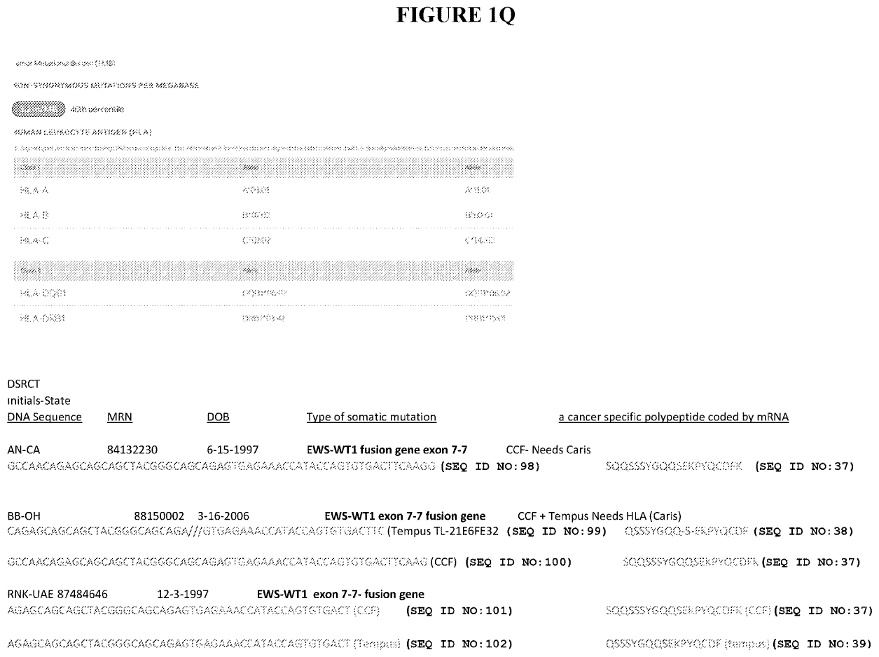

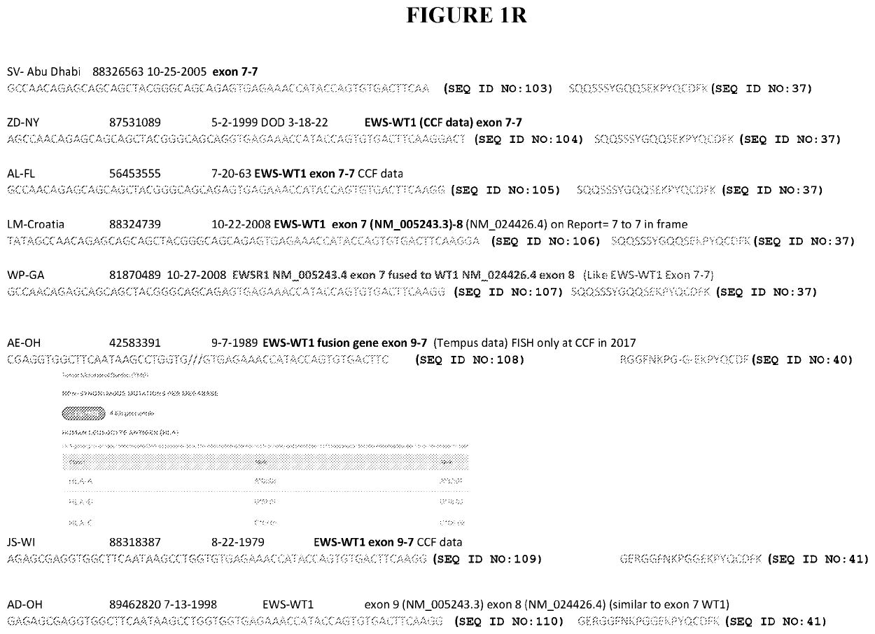

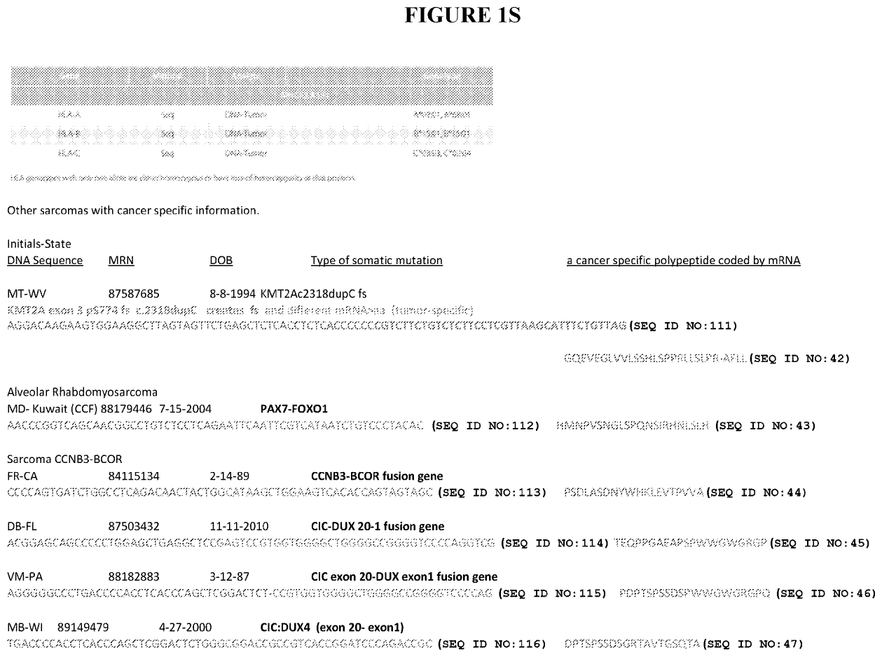

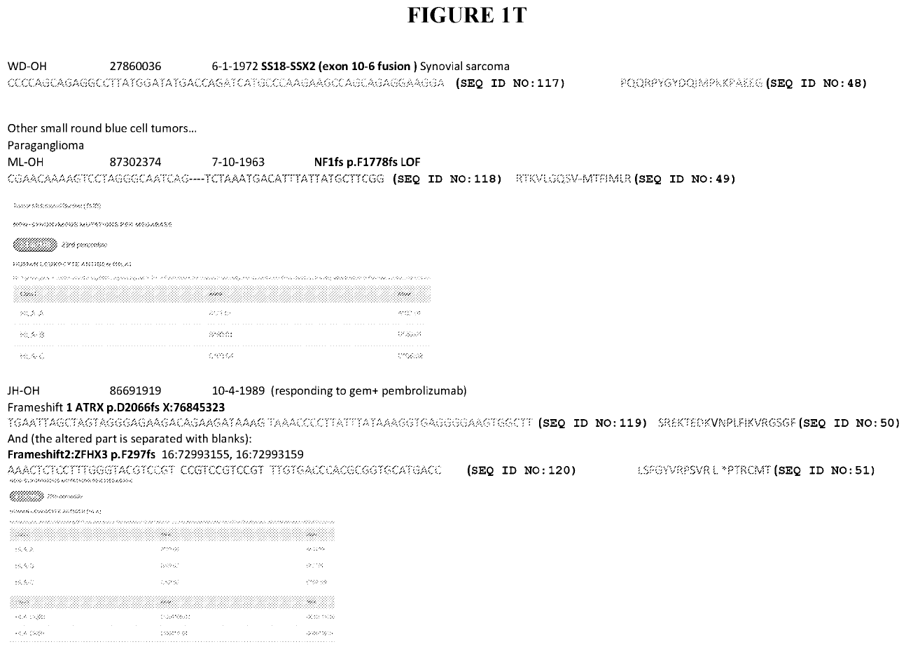

We detected fusions by analyzing large-scale tumor datasets including TCGA, Cosmic, FusionGDB. Fastaq files were mapped to the human reference genome (build hg38) followed by fusion calling using AGFusion software. We experimentally tested the binding capacity of peptides derived from EWSR1-FLI1 six different types, two types of EWSR1-ERG that is the second most common translocation in about 10%-15% of cases in EWS patients; FUS-ERG and EWSR1-WT1 proteins to the most common HLA types in population. We created stable single HLA expressing cell lines for all the picked common HLA alleles by transfecting HLA class I-negative K562 or C1R B cell lymphoblastoid cells and peptide stabilization of cell-surface HLA molecules was assessed using human single HLA class I allele transfected cells. Further we studied the immune-stimulating properties of positive binding peptides. After 21-27 days of coculturing healthy donor CD8 T cells with either autologous dendritic cells or single HLA expressing antigen presenting cells with each neoantigen fusion peptides and measured the antigen reactivity of Cytotoxic T (CTL) cells by ELISPOT. Based on the peptide binding and HLA specific CTL immunogenicity data, we have developed a library of targetable peptides for immunotherapy derived from Ewing sarcoma fusions (see FIG. 1 and Table 1). These peptides, and portions thereof, can be used for (1) cancer vaccines targeting the fusions, (2) TCR-like therapeutic antibodies that bind to MHC-fusion neopeptides, and (3) a CAR T against fusion peptide-MHC complex.

In certain embodiments, the fusion antigens herein are selected from the following: i) an EWS-FLi1 fusion protein that is an exon 7-exon 5, 6, 7, 8, or 10 exon fusion protein; ii) an EWS-FLi1 fusion protein that is an exon 10-exon 5, 6, or 7 exon fusion protein; iii) an EWS-FLi1 fusion protein that is an exon 9-exon 8 fusion protein; iv) an EWS-FLi1 fusion protein that is an intron 10-intron 4 fusion protein; v) an EWS-FEW fusion protein is an exon 7-exon 2 fusion protein; vi) an EWS-ERG fusion protein is an exon 7-exon 8 or 9 fusion protein; vii) an EWS-ERG fusion protein is an exon 10-exon 9 fusion protein; viii) an EWS-ERG fusion protein is an exon 9-exon 7 fusion protein; ix) an EWS-ATF fusion protein is an exon 8-exon 4 fusion protein; x) an EWS-WT1 fusion protein is an exon 7-exon 7 or 8 fusion protein; xi) an EWS-WT1 fusion protein is an exon 9-exon 7 fusion protein; xii) a CIC-DVX fusion protein is an exon 20-exon 1 fusion protein; and/or xiii) a SS18-SSX2 fusion protein is an exon 10-exon 6 fusion protein.

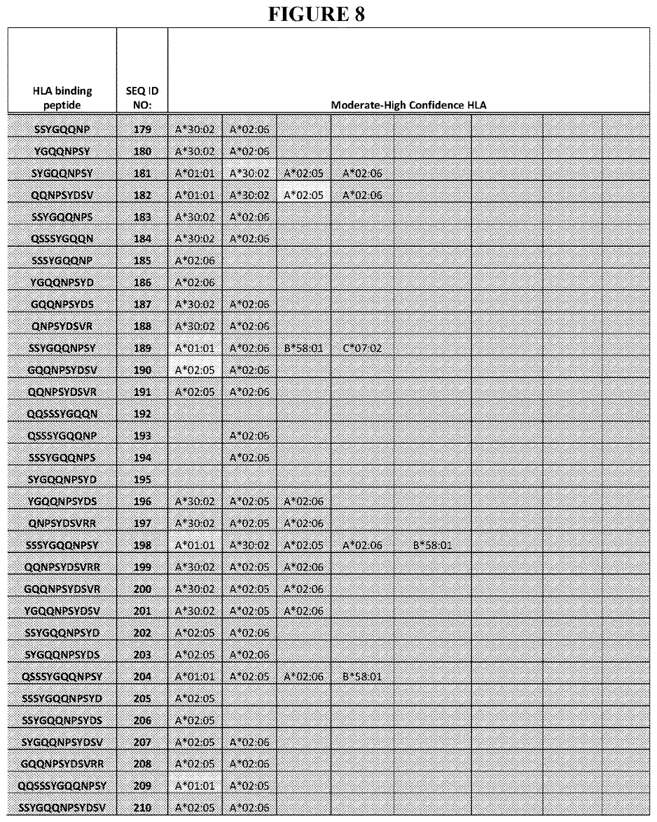

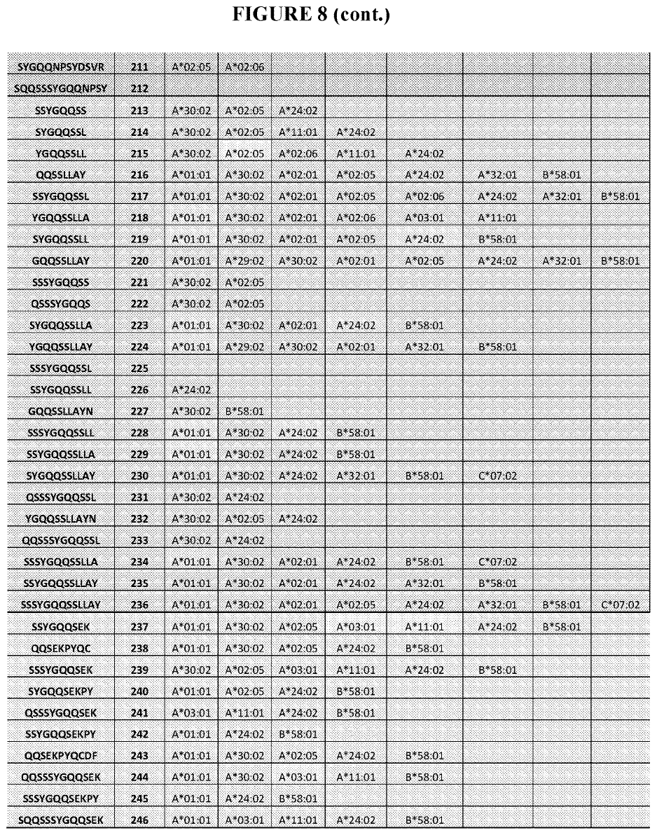

In certain embodiments, the fusion antigens herein are selected from any one or more of the peptides shown in Table 1 below or SEQ ID NOs: 179-246 from FIG. 8.

TABLE 1

| | | | SEQ ID | |
|---|---|---|---|---|
| | Fusion | Peptide | NO: | MS ID |
| EWSR1/FLI1 | EWSR1-FLI1_1109 | SYGQQNPSY | 121 | |
| TYPE 1 | EWSR1-FLI1_1109 | QQNPSYDSV | 122 | |
| FUSION | EWSR1-FLI1_1109 | SSYGQQNPSY | 123 | Peptide 6 |
| | EWSR1-FLI1_1109 | SSSYGQQNPSY | 124 | Peptide 3 |
| | EWSR1-FLI1_1109 | QSSSYGQQNPSY | 125 | Peptide 2 |
| EWSR1/FLI1 | EWSR1-FLI1_1121 | QQSSLLAY | 126 | |
| TYPE 2 | EWSR1-FLI1_1121 | SSYGQQSSL | 127 | |
| FUSION | EWSR1-FLI1_1121 | YGQQSSLLA | 128 | |
| | EWSR1-FLI1_1121 | SYGQQSSLL | 129 | |
| | EWSR1-FLI1_1121 | GQQSSLLAY | 130 | Peptide 1 |
| | EWSR1-FLI1_1121 | SYGQQSSLLA | 131 | |
| | EWSR1-FLI1_1121 | YGQQSSLLAY | 132 | Peptide 9 |
| | EWSR1-FLI1_1121 | SSSYGQQSSLL | 133 | |
| | EWSR1-FLI1_1121 | SSYGQQSSLLA | 134 | |
| | EWSR1-FLI1_1121 | SYGQQSSLLAY | 135 | Peptide 8 |
| | EWSR1-FLI1_1121 | SSSYGQQSSLLA | 136 | |
| | EWSR1-FLI1_1121 | SSYGQQSSLLAY | 137 | Peptide 7 |
| | EWSR1-FLI1_1121 | SSSYGQQSSLLAY | 138 | Peptide 5 |
| EWSR1/FLI1 | EWSR1-FLI1_1165 | YGQQNPYQI | 139 | |
| TYPE 3 | EWSR1-FLI1_1165 | SSYGQQNPY | 140 | |
| FUSION | EWSR1-FLI1_1165 | GQQNPYQIL | 141 | |
| | EWSR1-FLI1_1165 | SSSYGQQNPY | 142 | Peptide 4 |
| | EWSR1-FLI1_1165 | SYGQQNPYQI | 143 | |
| | EWSR1-FLI1_1165 | QSSSYGQQNPY | 144 | |
| | EWSR1-FLI1_1165 | SYGQQNPYQIL | 145 | |
| EWSR1/ERG | EWSR1-ERG_3030 | SYGQQNLPY | 146 | |
| | EWSR1-ERG_3030 | SSYGQQNLPY | 147 | |
| | EWSR1-ERG_3030 | SSSYGQQNLPY | 148 | |
| | EWSR1-ERG_3034 | QSSGQIQLW | 149 | |
| | EWSR1-ERG_3034 | QQSSGQIQL | 150 | |
| | EWSR1-ERG_3034 | GQQSSGQIQL | 151 | |
| | EWSR1-ERG_3034 | QQSSGQIQLW | 152 | |
| | EWSR1-ERG_3034 | YGQQSSGQIQL | 153 | |
| | EWSR1-ERG_3034 | GQQSSGQIQLW | 154 | |
| | EWSR1-ERG_3034 | QSSGQIQLWQF | 155 | |
| | EWSR1-ERG_3026 | KPGAPLPHL | 156 | |
| FUS/ERG | FUS-ERG_1 | FGGSGQIQL | 157 | |
| | FUS-ERG_1 | GGSGQIQLW | 158 | |
| | FUS-ERG_1 | FGGSGQIQLW | 159 | |
| | FUS-ERG_1 | KFGGSGQIQLW | 160 | |
| EWSR1/WT1 | EWSR1-WT1_46 | SSYGQQSEK | 161 | |
| | EWSR1-WT1_46 | QQSEKPYQC | 162 | |
| | EWSR1-WT1_46 | SSSYGQQSEK | 163 | |
| | EWSR1-WT1_46 | SYGQQSEKPY | 164 | |
| | EWSR1-WT1_46 | QSSSYGQQSEK | 165 | |
| | EWSR1-WT1_46 | SSYGQQSEKPY | 166 | |
| | EWSR1-WT1_46 | QQSEKPYQCDF | 167 | |
| | EWSR1-WT1_46 | QQSSSYGQQSEK | 168 | |
| | EWSR1-WT1_46 | SSSYGQQSEKPY | 169 | |
| | EWSR1-WT1_46 | SQQSSSYGQQSEK | 170 | |

In some embodiments, the fusion antigen polynucleotides or mRNAs herein comprise at least one chemical modification or chemically modified base, nucleoside, or nucleotide. The chemical modifications may comprise any modification which is not naturally present in said RNA or any naturally-occurring modification of adenosine (A), guanosine (G), uridine (U), or cytidine (C) ribonucleosides. For example, a single polynucleotide or mRNA may include both naturally-occurring and non-naturally-occurring modifications. Chemical modifications may be located in any portion of the polynucleotide or mRNA molecule and the polynucleotide or mRNA molecule may contain any percentage of modified nucleosides (1-100%, such as at least 20% . . . at least 40% . . . or at least 60%). In some embodiments, every particular base or nucleoside may be modified (e.g., every uridine is a modified uridine). In some embodiments, at least 20%, or 50%, or 80% of any single nucleotide (e.g., uracil) in the of the polynucleotide or mRNA is chemically modified. In some embodiments, a particular modification is used for every particular type of nucleoside or base (e.g., every uridine is modified to a 1-methyl-pseudouridine). Exemplary RNA modifications can be found in the RNA modification database (See, mods(dot)rna(dot)albany(dot)edu/home).

In some embodiments, the at least one chemical modification comprises a modified uridine residue. Exemplary modified uridine residues include, but are not limited to, pseudouridine, 1-methylpseudouridine, 1-ethylpseudouridine, 2-thiouridine, 4′-thiouridine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-0-methyl uridine.

In some embodiments, the at least one chemical modification comprises a modified cytosine residue. Exemplary nucleosides having a modified cytosine include 5-aza-cytidine, 6-aza-cytidine, pseudoisocytidine, 3-methyl-cytidine, N4-acetyl-cytidine, 5-formyl-cytidine, N4-methyl-cytidine, 5-methyl-cytidine, 5-halo-cytidine, 5-hydroxymethyl-cytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, lysidine, α-thio-cytidine, 2′-O-methyl-cytidine, 5,2′-O-dimethyl-cytidine, N4-acetyl-2′-O-methyl-cytidine, N4,2′-O-dimethyl-cytidine, 5-formyl-2′-O-methyl-cytidine, N4,N4,2′-O-trimethyl-cytidine, 1-thio-cytidine, 2′-F-aracytidine, 2′-F-cytidine, and 2′-OH-aracytidine.

In some embodiments, the at least one chemical modification comprises a modified adenine residue. Exemplary nucleosides having a modified adenine include 2-amino-purine, 2,6-diaminopurine, 2-amino-6-halo-purine, 6-halo-purine, 2-amino-6-methyl-purine, 8-azido-adenosine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenosine, 2-methyl-adenine, N6-methyl-adenosine, 2-methylthio-N6-methyl-adenosine, N6-isopentenyl-adenosine, 2-methylthio-N6-isopentenyl-adenosine, N6-(cis-hydroxyisopentenyl) adenosine, 2-methylthio-N6-(cis-hydroxyisopentenyl) adenosine, N6-glycinylcarbamoyl-adenosine, N6-threonylcarbamoyl-adenosine, N6-methyl-N6-threonylcarbamoyl-adenosine, 2-methylthio-N6-threonylcarbamoyl-adenosine, N6,N6-dimethyl-adenosine, N6-hydroxynoryalylcarbamoyl-adenosine, 2-methylthio-N6-hydroxynoryalylcarbamoyl-adenosine, N6-acetyl-adenosine, 7-methyl-adenine, 2-methylthio-adenine, 2-methoxy-adenine, α-thio-adenosine, 2′-O-methyl-adenosine, N6,2′-O-dimethyl-adenosine, N6,N6,2′-O-trimethyl-adenosine, 1,2′-O-dimethyl-adenosine, 2′-O-ribosyladenosine (phosphate), 2-amino-N6-methyl-purine, 1-thio-adenosine, 8-azido-adenosine, 2′-F-ara-adenosine, 2′-F-adenosine, 2′-OH-ara-adenosine, and N6-(19-amino-pentaoxanonadecyl)-adenosine.

In some embodiments, the at least one chemical modification comprises a modified guanine residue. Exemplary nucleosides having a modified guanine include inosine, 1-methyl-inosine, wyosine, methylwyosine, 4-demethyl-wyosine, isowyosine, wybutosine, peroxywybutosine, hydroxywybutosine, undermodified hydroxywybutosine, 7-deaza-guanosine, queuosine, epoxyqueuosine, galactosyl-queuosine, mannosyl-queuosine, 7-cyano-7-deaza-guanosine, 7-aminomethyl-7-deaza-guanosine, archaeosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methyl-inosine, 6-methoxy-guanosine, 1-methyl-guanosine, N2-methyl-guanosine, N2,N2-dimethyl-guanosine, N2,7-dimethyl-guanosine, N2,N2,7-dimethyl-guanosine, 8-oxo-guanosine, 7-methyl-8-oxo-guanosine, 1-methyl-6-thio-guanosine, N2-methyl-6-thio-guanosine, N2,N2-dimethyl-6-thio-guanosine, α-thio-guanosine, 2′-O-methyl-guanosine, N2-methyl-2′-O-methyl-guanosine, N2,N2-dimethyl-2′-O-methyl-guanosine, 1-methyl-2′-O-methyl-guanosine, N2,7-dimethyl-2′-O-methyl-guanosine, 2′-O-methyl-inosine, 1,2′-O-dimethyl-inosine, and 2′-O-ribosylguanosine (phosphate).

### II. Cancer Vaccine Adjuvants

In certain embodiments, the fusion neoantigen herein (see FIG. 1 and Table 1) are prepared in a composition (e.g., with a buffer or saline) that further contains a cancer vaccine adjuvant. Such preparations can be administered to cancer patients as cancer vaccines. The compositions are not limited by the type of adjuvant. Examples of such adjuvants include the following:
i) water-in-oil emulsions such as Montanide ISA 720 and Montanide ISA-51 adjuvants that contain Pathogen-associated molecular pattern molecules (PAMPs);
ii) Toll-like receptor (TLR) agonists such as polyinosinic-polycytidylic acid with polylysine and carboxymethylcellulose (Poly-ICLC); monophosphoryl lipid A (MPLA), imiquimod a TLR7 agonist, resiquimod, and CpG oligodeoxynucleotide (CpG ODN);
iii) Stimulator of interferon genes protein (STING) agonists;
iv) cytokines such as IL-2, IFN, IL-12, and granulocyte-macrophage colony stimulating factor (GM-CSF); and
v) a Modi-1 peptide vaccine that comprises two citrullinated vimentin peptides, as well as a citrullinated peptide from α-enolase, each peptide is conjugated to the TLR1/2 ligand adjuvant AMPLIVANT® (ISA Pharmaceuticals BV, Leiden, the Netherlands).

### III. Antibodies, and Fragments Thereof, to Fusion Neoantigens

In some embodiments, antibodies, and fragments thereof, specific to one of the fusion neoantigens herein (see FIG. 1, Table 1, and FIG. 8) are used for treating a patient with cancer. The antibodies may be prepared using the peptides or fragments thereof (e.g., spanning the fusion site) as shown in FIG. 1 and Table 1. Such antibodies include, but are not limited to polyclonal, monoclonal, chimeric, single chain, Fab fragments, and Fab expression libraries.

Various procedures known in the art may be used for the production of polyclonal antibodies directed against the fusion neoantigens herein. For the production of antibody, various host animals can be immunized by injection with the fusion neoantigen including but not limited to rabbits, mice, rats, sheep, goats, etc. In certain embodiments, the fusion neoantigen is conjugated to an immunogenic carrier (e.g., diphtheria toxoid, bovine serum albumin (BSA), or keyhole limpet hemocyanin (KLH)). Various adjuvants may be used to increase the immunological response, depending on the host species, including but not limited to Freund's (complete and incomplete), mineral gels (e.g., aluminum hydroxide), surface active substances (e.g., lysolecithin, pluronic polyols, polyanions, peptides, oil emulsions, keyhole limpet hemocyanins, dinitrophenol, and potentially useful human adjuvants such as BCG (Bacille Calmette Guerin) and Corynebacterium parvum).

For preparation of monoclonal antibodies directed toward the fusion neoantigens herein, it is contemplated, in certain embodiments, that any technique that provides for the production of antibody molecules by continuous cell lines in culture will find use with the present disclosure (See e.g., Harlow and Lane, Antibodies: A Laboratory Manual, Cold Spring Harbor Laboratory Press, Cold Spring Harbor, NY). These include but are not limited to the hybridoma technique originally developed by Köhler and Milstein (Köhler and Milstein, Nature 256:495 497 [1975]), as well as the trioma technique, the human B cell hybridoma technique (See e.g., Kozbor et al., Immunol. Tod., 4:72 [1983]), and the EBV hybridoma technique to produce human monoclonal antibodies (Cole et al., in Monoclonal Antibodies and Cancer Therapy, Alan R. Liss, Inc., pp. 77 96 [1985]).

In certain embodiments, monoclonal antibodies are produced in germ free animals utilizing technology such as that described in PCT/US90/02545. Furthermore, it is contemplated that human antibodies may be generated by human hybridomas (Cote et al., Proc. Natl. Acad. Sci. USA 80:2026 2030 [1983]) or by transforming human B cells with EBV virus in vitro (Cole et al., in Monoclonal Antibodies and Cancer Therapy, Alan R. Liss, pp. 77 96 [1985]).

In addition, it is contemplated that techniques described for the production of single chain antibodies (U.S. Pat. No. 4,946,778; herein incorporated by reference) will find use in producing fusion neoantigens herein (see FIG. 1 and Table 1) specific single chain antibodies. An additional embodiment of the invention utilizes the techniques described for the construction of Fab expression libraries (Huse et al., Science 246:1275 1281 [1989]) to allow rapid and easy identification of monoclonal Fab fragments with the desired specificity for the fusion neoantigens herein.

It is contemplated that any technique suitable for producing antibody fragments will find use in generating antibody fragments that contain the idiotype (antigen binding region) of the antibody molecule. For example, such fragments include but are not limited to: F(ab′)2 fragment that can be produced by pepsin digestion of the antibody molecule; Fab′ fragments that can be generated by reducing the disulfide bridges of the F(ab′)2 fragment, and Fab fragments that can be generated by treating the antibody molecule with papain and a reducing agent.

In the production of antibodies, it is contemplated that screening for the desired antibody will be accomplished by techniques known in the art (e.g., radioimmunoassay, ELISA (enzyme linked immunosorbant assay), “sandwich” immunoassays, immunoradiometric assays, gel diffusion precipitation reactions, immunodiffusion assays, in situ immunoassays (e.g., using colloidal gold, enzyme or radioisotope labels, for example), Western blots, precipitation reactions, agglutination assays (e.g., gel agglutination assays, hemagglutination assays, etc.), complement fixation assays, immunofluorescence assays, protein A assays, and immunoelectrophoresis assays, etc.

### IV. CAR T-Cells

Cart T-cells specific for one or more of the fusion neoantigens herein (see FIG. 1 and Table 1) can be made by methods that are known in the art (e.g., see U.S. Pat. Nos. 7,446,190; 8,399,645; and 7,741,46, all of which are herein incorporated by reference, and specifically for methods of making CAR T-cells using known antigens, such as those in FIG. 1, Table 1, and FIG. 8). In general, CAR T-cell are made by collecting T cells from the patient and re-engineering them in the laboratory to produce proteins on their surface called chimeric antigen receptors, or CARs. The CARs recognize and bind to specific proteins, or antigens, on the surface of cancer cells (such as those in FIG. 1, Table 1, FIG. 8, or fragments thereof). Exemplary methods of producing and using CAR T-cells are provided below.

The first step in the production of CAR T-cells is the isolation of T cells from human blood. CAR T-cells may be manufactured either from the patient's own blood, known as an autologous treatment, or from the blood of a healthy donor, known as an allogeneic treatment.

First, leukocytes are isolated using a blood cell separator in a process known as leukocyte apheresis. Peripheral blood mononuclear cells (PBMCs) are then separated and collected. The products of leukocyte apheresis are then transferred to a cell-processing center. In the cell processing center, specific T cells are stimulated so that they will actively proliferate and expand to large numbers. To drive their expansion, T cells are typically treated with the cytokine interleukin 2 (IL-2) and anti-CD3 antibodies.

The expanded T cells are purified and then transduced with a gene encoding the engineered CAR via a retroviral vector, typically either an integrating gammaretrovirus (RV) or a lentiviral (LV) vector. These vectors are very safe in modern times due to a partial deletion of the U3 region. The new gene editing tool CRISPR/Cas9 has recently been used instead of retroviral vectors to integrate the CAR gene into specific sites in the genome.

The patient undergoes lymphodepletion chemotherapy prior to the introduction of the engineered CAR T-cells. The depletion of the number of circulating leukocytes in the patient upregulates the number of cytokines that are produced and reduces competition for resources, which helps to promote the expansion of the engineered CAR T-cells.

### EXAMPLE

### Example 1

### Fusion Neoantigens as Targets for Cancer Immunotherapy

### Introduction

Ewing sarcoma (EWS) is the second most common type of soft tissue sarcoma with high mortality rates in pediatric patients and young adults. These tumors are most common in the second decade of life. The incidence in 10-19-year-olds is 9-10 per million per year. While rare, this is a classic pediatric cancer where outcomes are not optimal. Most studies support the origin of these tumors from mesenchymal stem cells (MSCs). Over the past 40 years, improvements in multi-agent chemotherapy approaches have increased the 5-year overall survival (OS) in patients without metastasis (1). However, 5-year OS remains less than 20% for those with metastatic EWS (2). Moreover, many patients will develop metastatic disease at some point, and once tumors recur, there are no effective therapeutic options. Patients with recurrent and/or refractory EWS have a dismal prognosis with a 5-year OS of 7-20% (3). Given the bleak long-term outcomes and very toxic therapies used (including surgery or radiation of the primary tumor site and multi-agent chemotherapy) there is an urgent need to explore and develop alternative treatment modalities including immunotherapies.

EWS harbor a limited number of genetic mutations and have a low mutation burden (4-6). However, over 95% of EWS are driven by a fusion oncogene (7). Fusion genes are formed by combining parts of two different genes into a novel one that typically encodes for an in-frame chimeric protein, including key domains specified by the parental genes. Gene fusions occur in all types of human malignancies, and many recurrent fusions are established to be strong drivers of tumorigenesis and progression. Previously, our group showed that fusion neoantigens from head and neck squamous cell carcinoma can drive tumor clearance via T-cell mediated mechanisms (8), supporting the hypothesis that novel therapies targeting these driver fusions in cancer have achieved outstanding outcomes (9, 10). In EWS, EWSR1-FLI1 is the most common fusion oncogene being found in 80-90% of EWS and has at least 11 subtypes exist in which exons 7, 9, or 10 from EWSR1 are combined with exons 4, 5, 6, 7, or 8 from FLI1. Although the breakpoint sequences of each subtype are different, EWS-FLI1 type 1 fusions (EWSR1 exon 7 fused to FLI-1 exon 6) are present in approximately 60% of patients with EWS tumors. The EWSR1-FLI1 fusion has been demonstrated to lead to tumorigenesis and is crucial to maintaining the malignant phenotype. Additional fusions in EWS include EWSR1-ERG, EWSR1-ERT, EWSR1-WT, and FUS-ERG. The altered amino acid sequences at the break/fusion point of these fusion oncoproteins represent neoantigens that can be presented by HLA molecules to evoke an immune response that selectively targets fusion oncoprotein containing cells. These fusion neoantigens are not found in normal cells; thus, immunotherapies targeting these fusions would be specific to tumor cells and predicted to provide clinical benefit to more than 90% of patients with EWS.

### Results

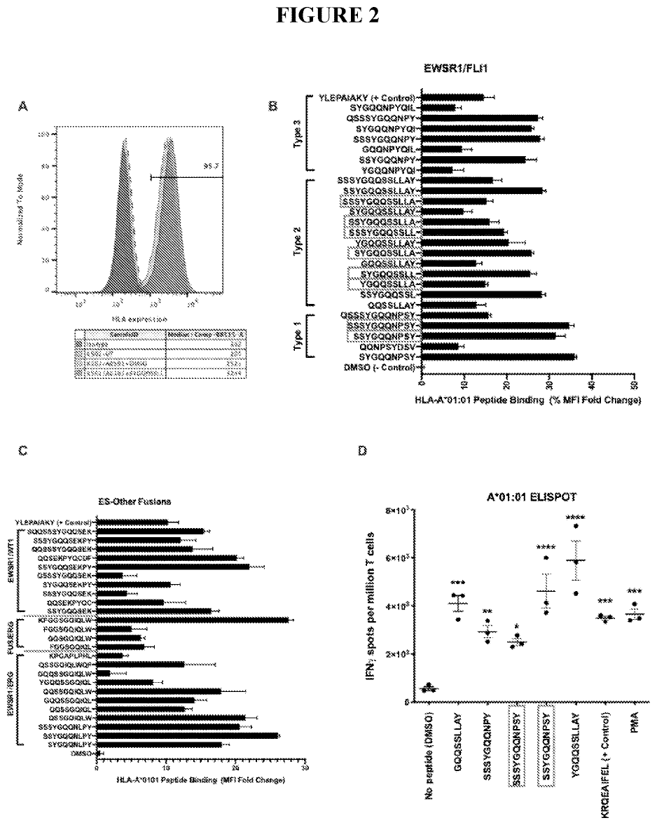

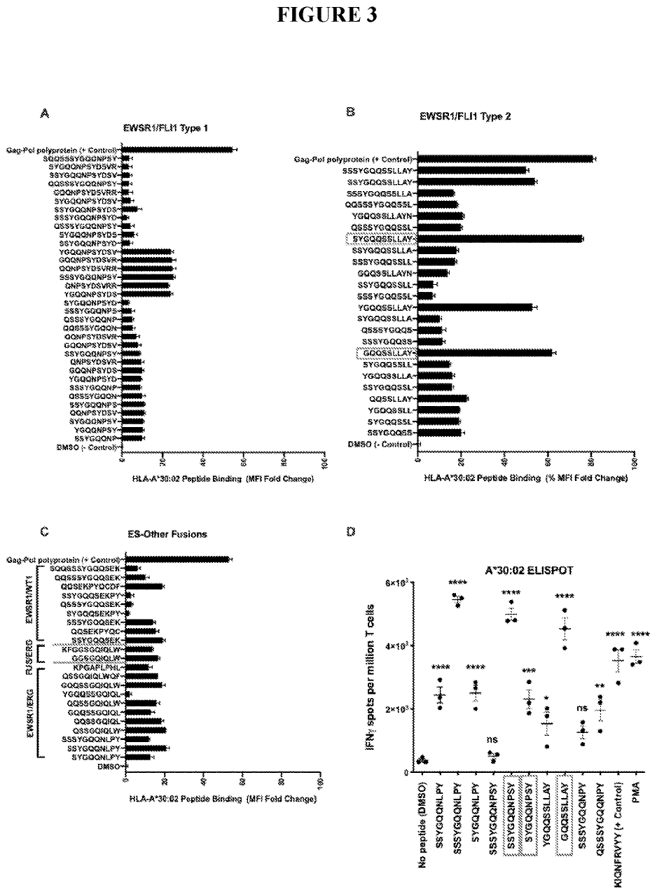

In this example we detected fusions by analyzing large-scale tumor datasets including TCGA, Cosmic, FusionGDB. Fastaq files were mapped to the human reference genome (build hg38) followed by fusion calling using AGFusion software. Since out of frame fusions (breakpoint at UTR, intron or non-coding RNA) are not functional fusion proteins based on the reference coding region annotations, they were filtered out to reduce false positives in predicting fusion neoantigens. For each predicted fusion, we obtained the translated protein sequence output by AgFusion and constructed 8-14 mers peptides. By using ~3000 human class I HLA sequences, pMHC binding affinity and binding affinity percent rank were predicted by NetMHCpan version 4.0 in binding affinity mode with other parameters set as default. Peptides with binding affinity percent rank <=2 are reported as candidate neoantigens. This identified 44626 peptide/HLA combinations with predicted strong interactions. To validate binding we experimentally tested the binding capacity of peptides derived from EWSR1-FLI1. We examined. three types of EWSR1-ERG, which is the second most common translocation at about 10%-15% of cases in EWS patients; We also examined FUS-ERG and EWSR1-WT1 fusion proteins in the context of the most common HLA types in the USA population. We created stable monoallelic HLA expressing cell lines for all the picked common HLA alleles by transfecting HLA class I-negative K562 or C1R B cell lymphoblastoid cells. Peptide stabilization of cell-surface HLA molecules was assessed using human single HLA class I allele transfected cells. To date, we investigated the binding of 50 EWS neoantigen peptides to 13 of the most common HLAs, which confirmed strong binding in 134 peptide/HLA combination (FIG. 2-B,C, FIG.-3B,C). Of the peptide/HLA combinations with predicted binding score less than 0.5, binding was confirmed in 50 of 54 (93%), while 43 of 63 (68%) of the peptide/HLA combinations with predicted binding score 0.5-2 were confirmed.

Furthermore, we studied the immune-stimulating properties of positive binding peptides. After 21-27 days of co-culturing healthy donor CD8⁺ T cells with either autologous dendritic cells or single HLA expressing antigen presenting cells with each neoantigen fusion peptides, we measured the antigen reactivity of CD8⁺ cytotoxic T (CTL) cells by ELISPOT. Based on the peptide binding and HLA specific CTL immunogenicity data, we have found targetable peptides for immunotherapy derived from Ewing sarcoma fusions (FIG. 2C). To date we have tested 30 neoantigen-peptide/HLA combinations for immunogenicity confirming 21 (70%) are immunogenic (FIG. 2D, 3D). These represent validated targets that can be used in the setting of correct HLA genotypes of the patient under consideration.

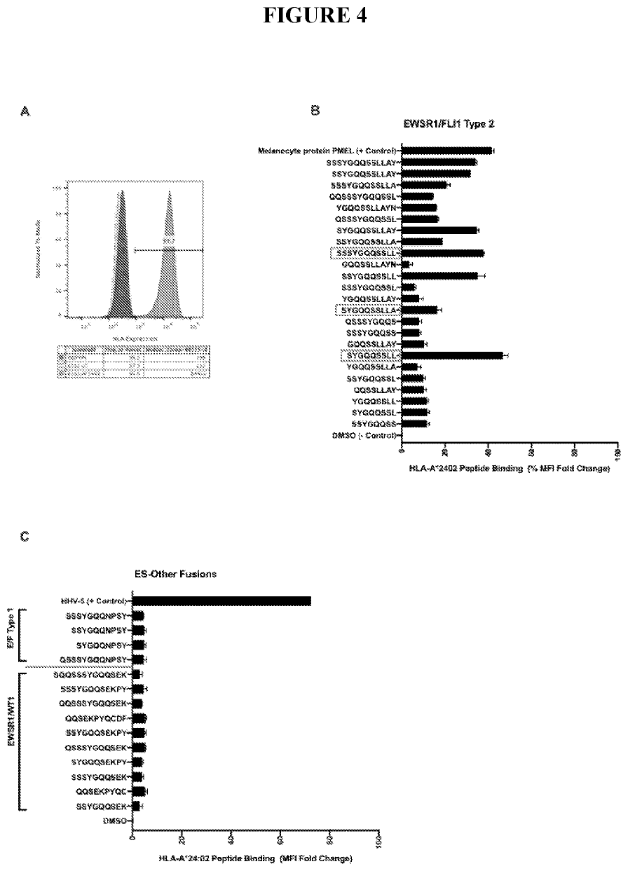

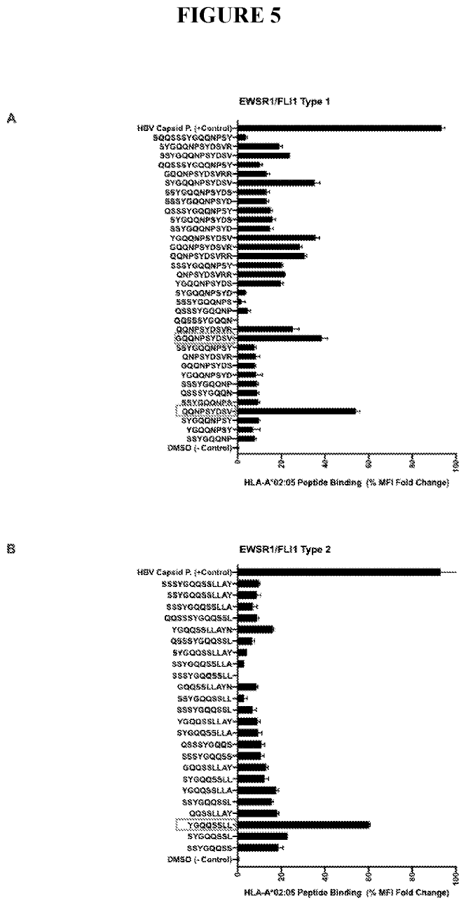

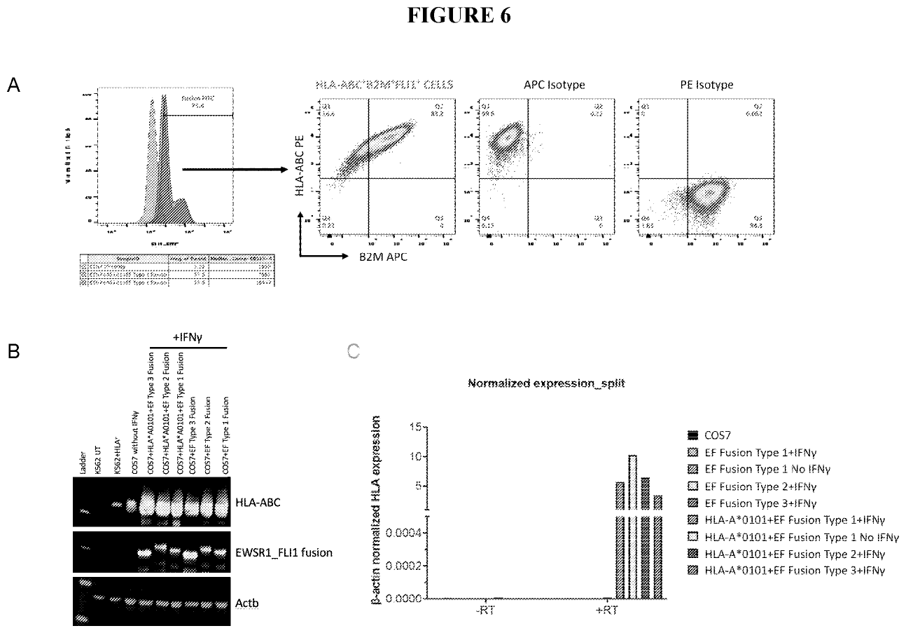

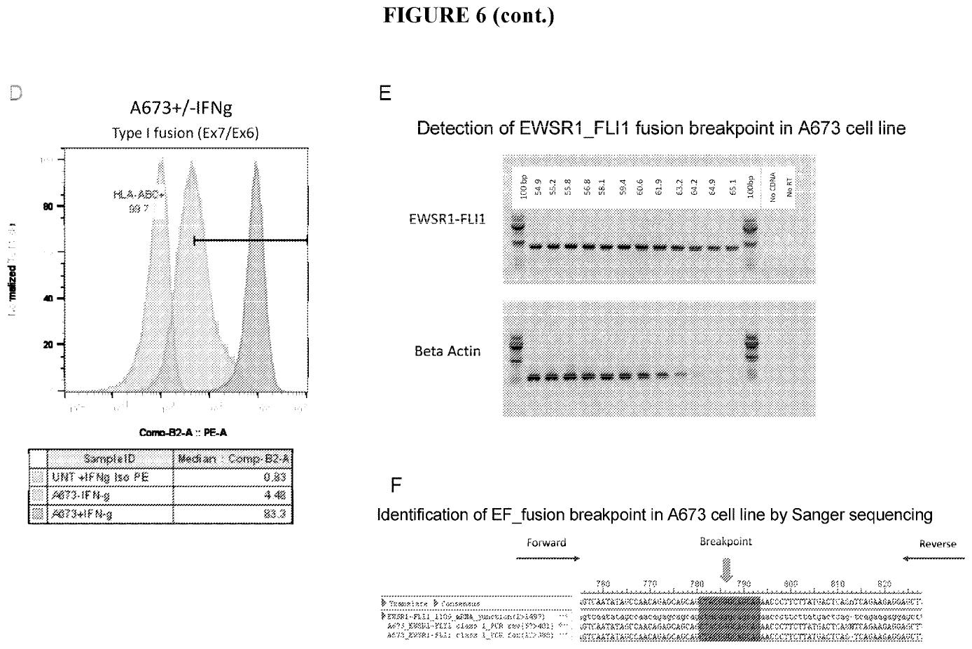

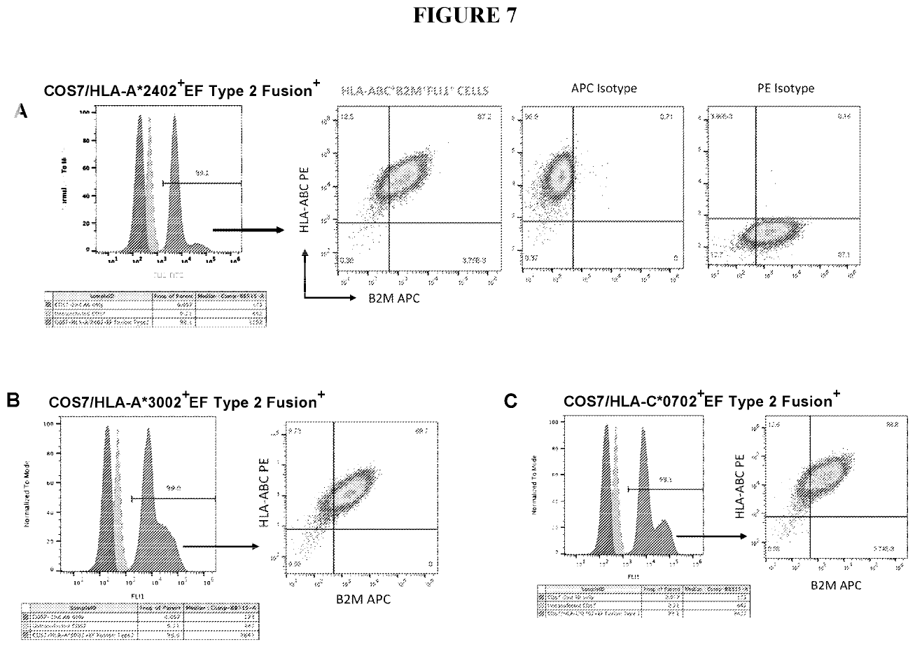

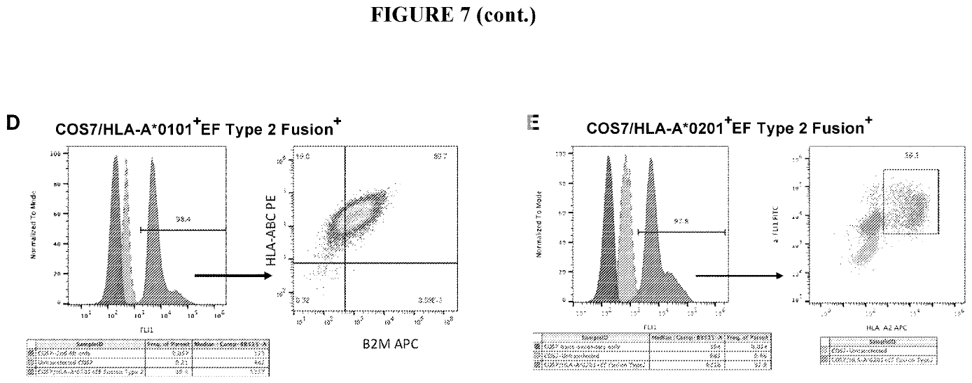

To confirm proper processing and presentation of the candidate fusion neoantigen peptides from the full length fusion oncoprotein, we utilize nano LC-MS/MS to identify peptides derived through proteolytic processing within the cell and presented by specific HLA. EWS cell lines harboring the fusion commonly found in EWS and HLA are tested to further confirm targets. For peptides/HLA combination without an appropriate EWS cell line, we performed mass spectrometry on cell lines engineer to expression the full length fusion oncoprotein and HLA allele to be tested. We show examples where we have used mass spectrometry of COS7 cells expressing 3 different EWSR1-FLI1 fusion with to 2 HLA alleles, showing six peptides that are properly processed and presented (FIG. 4).

For immunotherapies targeting these tumor specific fusions to be clinical actionable the fusion neoantigen found in the tumor needs to bind the specific HLA expressed by the patient. To investigate this, we have begun determining the HLA haplotype and fusion oncoprotein patterns in CCF EWS patients to confirm EWS patients have HLAs binding to the neoantigen peptides produced by fusion oncoprotein present in the tumor. To date 8 patients have been studied and nearly all patients were found to harbor multiple HLA alleles that bound to two or more fusion neoantigen peptides. In addition, we are putting together a large cohort of EWS cancer patients from public dataset to further confirm the frequency of fusion neoantigen/HLA binding combination in EWS patients

### Materials and Methods:

### Cell Lines:

COS-7, K562, A673, RD-ES, SKES-1, SK-PN-DW cells were purchased from American Type Culture Collection (ATCC). TCC-466, EWS-1, EWS-2, EWS-3, XT-EWS-3 were gifts from Translational Hematology & Oncology Research, Lerner Research Institute, Cleveland Clinic. COS-7, K562, A673, RD-ES, SK-PN-DW were culture in IMDM (Thermo Fisher Scientific, 31980030) with 10% FBS and 1% penicillin-streptomycin. SKES-1 was cultured in ATCC-formulated McCoy's 5a Medium Modified with 15% FBS and 1% penicillin-streptomycin. EWS-1, EWS-2, EWS-3, XT-EWS-3 were cultured in DMEM (high glucose, pyruvate, Thermo Fisher Scientific, 11995065) with 10% FBS, 0.1 mM MEM non-essential amino acids (Thermo Fisher Scientific, 11140050), 1% penicillin-streptomycin. TCC-466 were cultured in RPMI-1640 with 10% FBS and 1% penicillin-streptomycin. Human PBMCs with defined HLA-type were purchased from CTL immunospot. CD3+ T cells were sorted from PBMCs. T cells were cultured in IMDM with 10% FBS, 1% penicillin-streptomycin, 100 IU/ml recombinant human IL-2, and 5 ng/ml recombinant human IL-7. All cells were grown at 37° C. in 5% CO2 with humidification.

### Generation of Stable Single HLA Expressing Cell Lines

K562 cells were transfected using Nucleofection with program FF-120 and 10 μg of linearized HLA plasmid in Single Nucleocuvette. Single clones were selected against 1 mg/ml G418 for 14 days, stained with anti-HLA-ABC, anti-B2M antibodies and sorted single cells per well in 96 well plates by FACSAria Fusion. The resistant clones were expanded in 10% FBS containing IMDM/Glutamax and used in further assays. K562/HLA-A*0101, HLA-A*0201, HLA-A*2402, HLA-A*2902, HLA-A*3002, HLA-A*3201, HLA-A*2601, HLA-A*0205, HLA-A*0206, HLA-A*0301, HLA-A*1101, HLA-B*0702, HLA-B*0801 HLA-B*4402, HLA-B*4403, HLA-B*3501, HLA-B*5301, HLA-B*5801, HLA-C*0702, and HLA-C*0501 cell lines were generated.

### Peptide Binding and Stabilization Assay

Monoallelic 2×10⁵ HLA-expressing K562 cells, were resuspended in 200 μl 10% FBS containing IMDM, and incubated with 50 μM of the indicated peptides for 16 h at 26° C. and additional 2 h at 37° C. in 96-well plates. All peptides were synthesized at a purity of >90% by Genescript and dissolved in DMSO (Sigma Aldrich). Cells were stained with Zombie Violet, anti-HLA and anti-β2M antibodies. Cell surface HLA and B2M expression were analyzed by flow cytometry (Symphony, BD). ([mean fluorescence intensity (MFI)ₛₐₘₚₗₑ−MFI_{background}]/MFI_{background})*100 formula used to calculate MFI fold change. MFI_{background} represents the value without peptide in the presence of same volume of DMSO.

### Flow Cytometric Analysis

For flow cytometry analysis, the antibodies used are listed with the supplier, catalogue number, and dilution: PE-anti-HLA-A2 clone: BB7.2 (BD, cat #343306, 1:100), Alexa Fluor 405 anti-CD3e clone: UCHT1 (Thermo Fisher, cat #CD0326, 1:100), PerCP/Cy5.5 anti-CD4 clone: A161A1 (Biolegend, cat #357414, 1:100), APC-H7 anti-CD8 clone: SKI (BD, cat #560179, 1:100), PE/Dazzle 594 anti-CD137 clone: 4B4-1 (Biolegend, cat #309825, 1:100), Violet Live/Dead (Biolegend, cat #423114, 1:1000), PE/APC anti-HLA-ABC clone: W6/32 (Biolegend, cat #311410, 1:50), anti-B2M clone: 2M2 (Biolegend cat #316304, 1:100), Alexa Fluor® 488 anti-rabbit IgG (H+L) secondary (Cell signaling, cat #44112s, 1:1000), anti-FLI1 clone: EPR4646 (Abcam, cat #ab133485, 1:1000). For extracellular staining, cells were stained with Zombie Violet live/dead stain (Invitrogen) for 15 min at room temperature. Then, cells were washed and blocked with Fc receptor blocking solution (Biolegend) for 10 min and stained with fluorochrome labeled antibodies for 30 min in the dark at 4° C. For intracellular staining, cells were permeabilized with Fixation/Permeabilization kit (eBioscience) according to the manufacturer's instructions. Cells were then incubated antibodies prepared in 1× Perm/Wash buffer (eBioscience) for 30 minutes, and then washed twice in Perm/Wash buffer. Next, cells were incubated in secondary antibody if the primary antibody anconjugated for 30 min, and washed twice in Perm/Wash buffer prior to data collection. Samples were collected on Symphony (BD Biosciences), and compensation and data analysis were carried out with FlowJo vX.0.7 (BD Biosciences). For t-distributed stochastic neighbor embedding analysis, precompensated and gated data were exported from FlowJo.

### Induction of Neoantigen-Specific Cytotoxic T Cells.

HLA typed healthy donor PBMCs were obtained from CTL Immunospot. CD8+ T cells were isolated from PBMCs using Dynabeads CD8 Positive Isolation Kit (Thermo Fisher Scientific). CD8⁻ cells were used to generate monocyte-derived dendritic cells using plastic adherence methods, and were cultured in AIM-V media containing 2% human AB serum, 500 U/ml rhIL-4 (R&D Systems), and 1000 U/ml rhGM-CSF (R&D Systems) for 5 days. B lymphocytes were isolated from CD8 fraction of PBMCs by CD19 Positive Selection Kit (Stem cell). Isolated B cells were plated in IMDM/Glutamax medium supplemented with 10% human AB serum, 2 ng/ml rhIL-4 (R&D Systems), 5 μg/ml insulin (Sigma-Aldrich) and 2 μg/ml Human CD40 Ligand/TNFSF5 Protein, Fc Tag-active trimer (ACRO-Biosystems). On day 5, half of the medium was removed and replaced with medium supplemented with fresh recombinant CD40 ligand (rCD40L) and IL-4. At day 7 and day 10, the expanding CD40-B cells were counted, cell density was adjusted and cells were cultured for further expansion and maintained until day 14. To activate T cells, autologous dendritic cells, B cells or single HLA expressing K562 cells pulsed with 50 μM peptides for 4-12 hours and irradiated 30 Gy for autologous cells and 150 Gy for K562 cells. CD8+ T cells were cocultured with antigen presenting cells with 1:1,1:4 or 1:10 ratio depending on cell types in the presence of 30 IU/ml of IL-2, 50 ng/ml IL-7, 50 ng/ml IL-15 and, 30 ng/ml IL-21 in 10% FBS containing IMDM/Glutamax. IL-2, IL-7, IL-15 were replenished every 2-3 days by replacing half of the media. Peptide pulsed irradiated antigen pulsed APCs were added every 5 days of culture. After 15 days of coculture, cells were collected and stained with anti-CD3, anti-CD8, and anti-CD137 antibodies for at 4° C., and cells were washed once prior to acquisition. Zombie Violet negative CD3+CD8+CD137+ cells were sorted using BD FACSAria Fusion cell sorter. Cells were cultured additional 7-10 days with peptide pulsed APCs in presence of cytokines.

### Enzyme-Linked Immunospot (ELISPOT) Assay

Interferon IFN-γ secretion of T cells were detected by ELISPOT using Human IFN-γ single color ELISPOT procoated plates (CTL-Immunospot) according to the manufacturer's instructions. Briefly, single HLA expressing K562 or C1R cells were pulsed with each respective peptides before co-culture for 16 h at 37° C., 5% CO2. T cells were pre-treated with 30 IU/ml IL-2 for 16 h at 37° C. T cells (5×10⁴ cells) co-cultured with the peptide-pulsed irradiated K562 or C1R cells (2×10⁴ cells) at 37° C. for 20-24 h in serum free CTL-Test medium (CTL-Immunospot) at 37° C. in ELISPOT plates. Spots were captured and analyzed by an automated ELISPOT reader, ImmunoSPOT S4 and the ImmunoSpot Professional Software package, Version 5.1 (CTL-Immunospot). Statistical testing was performed using the two-tailed t-test.

### Western Blot Analysis

For immunoblotting, cells were washed with PBS and lysed in CelLytic™ MT Cell Lysis Reagent (Sigma, C3228) containing Protease Inhibitor cocktail (Thermofisher, 78430). Protein concentrations were measured by using the BCA Protein Assay (Thermofisher, 23225). The reactions were stopped by the addition of SDS sample buffer, and proteins were resolved by SDS-PAGE gels and immunoblotted with anti-HLA-ABC (Clone: EMR8-5, ab70328) and anti-Fli1 (clone: EPR4646, ab133485) antibodies.

### Quantitative Reverse Transcription PCR

Total RNA was isolated from HLA and fusion plasmid transfected COS7 cells with the RNeasy Mini Kit (Qiagen, 74106). Complementary DNA (cDNA) was synthesized with SuperScript IV Reverse Transcriptase (Thermo, 8090050) according to the manufacturer's protocol. All quantitative real-time PCRs (qRT-PCRs) were performed with a Real-Time PCR System and Quanti Studio 3 (Applied Biosystems) using PowerUp SYBR Green Master Mix (Applied Biosystems, A25742) and custom-designed primers in a final volume of 10 μl. Cycling conditions were 98° C. for 2 min, followed by 30 cycles of 98° C. for 10 s and 60° C. for 30 s. Relative expression of target genes was measured using the comparative ΔΔCt method, and ACTB was used as internal control. The sequences of the qRT-PCR primers are:

| Target | Oligos |
|---|---|
| EWS-FLI1 | F: cagccagtcactgcacctc (SEQ ID NO: 171) |
| breakpoint | R: gcttccagggttggctagg (SEQ ID NO: 172) |
| HLA-A*0101 | F: tccggagaggagcagagata (SEQ ID NO: 173) |
| | R: atgctgcctgggtatagga (SEQ ID NO: 174) |
| HLA-A*2402 | F: cgccctgcggtactataatc (SEQ ID NO: 175) |
| | R: tgcctcccactttctctttg (SEQ ID NO: 176) |
| ActB | F: gcaagagaggcattctcacc (SEQ ID NO: 177) |
| (Monkey) | R: ggggtgttgaaggtctcaaa (SEQ ID NO: 178) |

### Mass Spectrometry

EWS cell lines harboring fusion oncoprotein and HLA of interested were cultured until ~90% confluent, cultures washed with 1×PBS, incubated with Accutase (StemCell) for 10 min and collected in a 50-mL centrifuge tube. Cells were centrifuged at 300 g for 5 min and the supernatant discarded. Cell pellets were snap frozen in liquid nitrogen and stored at −80° C. COS7 cells were co-transfected with fusion and HLA plasmids using Lipofectamine 3000 transfection reagent (Thermo Fisher Scientific) using the manufacturer's protocols. Plasmids for eukaryotic cell line transfections encoding fusion proteins and HLA class I alleles were synthesized and cloned into pcDNA3.1/hygro(−) and pcDNA3.1, respectively by Genescript. COS-7 transfection and HLA IP were performed as previously described (11). Briefly, COS-7 cells seeded into 150 cm plates were transfected at 90% confluency using Lipofectamine 3000 Reagent (Thermo Fisher Scientific). For each plate, 76 μg of plasmids (30.4 μg of HLA plasmid and 45.6 μg of fusion plasmid) were mixed with 75 μL of Lipofectamine P3000 in 1.9 mL of Opti-MEM (Thermo Fisher Scientific). In a separate tube, 75 μL of Lipofectamine 3000 Reagent was mixed with 1.9 mL of Opti MEM. The contents of the two tubes were mixed and allowed to complex for 10 min. Medium floating cells were removed and 50 mL of fresh complete medium was added followed by the Lipofectamine-DNA mixture. Plates were gently swirled to mix reagents with the media. Cells were grown to near confluency in 150 cm plates. Cells were harvested 48 hr post-transfection. Cultured cells were washed with PBS two times and cells were treated with accutase for 10 min and collected in a 50-mL centrifuge tube. Small aliquots of cells were collected for flow cytometry, quantitative RT-PCR, and Western blotting to confirm successful transfection. The reminder of the cells were centrifuged at 300×g for 5 min and the supernatant discarded. Cell pellets were snap frozen in liquid nitrogen and stored at −80° C. The cell pellets were shipped to Creative proteomics, MS Bioworks or Complete Omics Inc for HLA immunoprecipitation, peptide elution and LC MS/MS analysis.

We have identified multiple recurrent neoantigens from the driver fusions in Ewing's sarcoma. Our in silco analysis of fusion neoantigen produced by the fusion oncogene common in EWS identified 44,626 neoantigen peptide/HLA combinations with strong binding. We have assembled a broad list of prioritized neoantigen peptides that are targetable, spanning the most common HLA genotypes in the population (see, e.g., Table 1). In vitro HLA/peptide binding assay confirm binding in a useful subset of peptide/HLA combinations tested.

T cell assays from patient-derived PBMCs were used to measure the immunogenicity of each neoantigen. To date we have analyzed 30 neoantigen peptide/HLA combinations for immunogenicity and found T cell activation in 21. In summary, we developed a well characterized bank of neoantigens produced by fusion oncoproteins common in EWS that span commonly represented HLA genotypes-enabling an off-the-shelf resource for targetable neoantigens.

### REFERENCES

1. Esiashvili N, Goodman M, Marcus R B, Jr. Changes in incidence and survival of Ewing sarcoma patients over the past 3 decades: Surveillance Epidemiology and End Results data. J Pediatr Hematol Oncol. 2008; 30 (6): 425-30.
2. Van Mater D, Wagner L. Management of recurrent Ewing sarcoma: challenges and approaches. Onco Targets Ther. 2019; 12:2279-88. Epub 2019 Apr. 17.
3. Fayzullina D, Tsibulnikov S, Stempen M, Schroeder B A, Kumar N, Kharwar R K, Acharya A, Timashev P, Ulasov I. Novel Targeted Therapeutic Strategies for Ewing Sarcoma. Cancers (Basel). 2022; 14 (8). Epub 2022 Apr. 24.
4. Dharia et al., A first-generation pediatric cancer dependency map. Nat Genet. 2021; 53 (4): 529-38. Epub 2021 Mar. 24.
5. Crompton et al., The genomic landscape of pediatric Ewing sarcoma. Cancer Discov. 2014; 4 (11): 1326-41. Epub 2014 Sep. 5.
6. Tirode et al., St. Jude Children's Research Hospital-Washington University Pediatric Cancer Genome P, the International Cancer Genome C. Genomic landscape of Ewing sarcoma defines an aggressive subtype with co-association of STAG2 and TP53 mutations. Cancer Discov. 2014; 4 (11): 1342-53. Epub 2014 Sep. 17.
7. Riggi N, Suva M L, Stamenkovic I. Ewing's Sarcoma. N Engl J Med. 2021; 384 (2): 154-64. Epub 2021 Jan. 27.
8. Yang et al., Immunogenic neoantigens derived from gene fusions stimulate T cell responses. Nat Med. 2019; 25 (5): 767-75. Epub 2019 Apr. 24.
9. Evans C H, Liu F, Porter R M, O'Sullivan R P, Merghoub T, Lunsford E P, Robichaud K, Van Valen F, Lessnick S L, Gebhardt M C, Wells J W. EWS-FLI-1-targeted cytotoxic T-cell killing of multiple tumor types belonging to the Ewing sarcoma family of tumors. Clin Cancer Res. 2012; 18 (19): 5341-51. Epub 2012 Aug. 11.
10. Wang Y, Shi T, Song X, Liu B, Wei J. Gene fusion neoantigens: Emerging targets for cancer immunotherapy. Cancer Lett. 2021; 506:45-54. Epub 2021 Mar. 7.
11. Wang Q, Douglass J, Hwang M S, Hsiue E H, Mog B J, Zhang M, Papadopoulos N, Kinzler K W, Zhou S, Vogelstein B. Direct Detection and Quantification of Neoantigens. Cancer Immunol Res. 2019; 7 (11): 1748-54. Epub 2019 Sep. 19.

All publications and patents mentioned in the specification and/or listed below are herein incorporated by reference. Various modifications and variations of the described method and system of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the relevant fields are intended to be within the scope described herein.

1. A method of treating a patient with cancer comprising:
treating a patient that has cancer with a composition comprising:
a) at least a portion of at least one fusion neoantigen selected from: i) an EWSR1-FLi1 fusion protein, ii) an EWS-FEV fusion protein; iii) an EWSR1-ERG fusion protein, iv) an EWS-ATF fusion protein; v) an EWS-WT1 fusion protein; vi) a KMT2A c2318 dupC fusion protein; vii) a PAX7-FOXO1 fusion protein; viii) a CCNB3-BCOR fusion protein; ix) CIC-DVX fusion protein; x) SS18-SSX2 fusion protein; xi) a FN1fs p.F1778 fusion protein; xii) 1 ATRX pD20GG fusion protein; and xiii) frameshift 2:2FHX3 p.F297 fusion protein;
b) a nucleic acid sequence encoding said at least a portion of said at least one fusion neoantigen;
c) an antibody, or antigen-binding fragment of said antibody, the specifically binds said at least a portion of said at least one fusion neoantigen; and/or
d) a chimeric antigen receptor T-cell (CAR T cell) that expresses a chimeric antigen receptor that is specific for said at least a portion of said at least one fusion neoantigen.

2. The method of claim 1, wherein said at least one fusion neoantigen is selected from any of SEQ ID NOs: 1-51, 121-170, and 179-246, and/or wherein said nucleic acid sequence encoding said at least a portion of said at least one fusion neoantigen is selected from SEQ ID NOs: 52-120.

3. The method of claim 1, wherein said at least a portion of said at least one fusion neoantigen comprises: i) at least a portion of said EWSR1 and said FLi1 proteins; ii) at least a portion of said EWSR1 and said ERG proteins; iii) at least a portion of said FUS and said ERG proteins; or said EWSR1 and said WT1 proteins.

4. The method of claim 1, wherein:
i) said EWS-FLi1 fusion protein is an exon 7-exon 5, 6, 7, 8, or 10 exon fusion protein;
ii) said EWS-FLi1 fusion protein is an exon 10-exon 5, 6, or 7 exon fusion protein;
iii) said EWS-FLi1 fusion protein is an exon 9-exon 8 fusion protein;
iv) said EWS-FLi1 fusion protein is an intron 10-intron 4 fusion protein;
v) said EWS-FEW fusion protein is an exon 7-exon 2 fusion protein;
vi) said EWS-ERG fusion protein is an exon 7-exon 8 or 9 fusion protein;
vii) said EWS-ERG fusion protein is an exon 10-exon 9 fusion protein;
viii) said EWS-ERG fusion protein is an exon 9-exon 7 fusion protein;
ix) said EWS-ATF fusion protein is an exon 8-exon 4 fusion protein;
x) said EWS-WT1 fusion protein is an exon 7-exon 7 or 8 fusion protein;
xi) said EWS-WT1 fusion protein is an exon 9-exon 7 fusion protein;
xii) said CIC-DVX fusion protein is an exon 20-exon 1 fusion protein; and/or
xiii) said SS18-SSX2 fusion protein is an exon 10-exon 6 fusion protein.

5. The method of claim 1, wherein said antibody, and said antigen-binding fragment of said antibody, specifically bind said at least a portion of said at least one fusion neoantigen when it is in a complex with a major histocompatibility complex molecule.

6. The method of claim 1, wherein said CAR T-cell specifically binds said at least a portion of said at least one fusion neoantigen when it is in a complex with a major histocompatibility complex molecule.

7. The method of claim 1, further comprising determining if a sample collected from said patient is positive or negative for said at least one fusion neoantigen or nucleic acid encoding said at least one fusion neoantigen.

8. The method of claim 7, wherein said determining comprises performing an immuno-assay on said sample, or derivative thereof, to determine the presence or absence of said at least one fusion neoantigen.

9. The method of claim 7, wherein said determining comprises performing a nucleic acid assay on said sample, or a derivative thereof, to determine the presence or absence of said at least one fusion neoantigen.

10. The method of claim 1, further comprising: testing a sample from said patient to determine at least one HLA type for said patient, and/or receiving results of testing said sample for said at least one HLA type of said patient, and wherein said at least a portion of said at least one fusion neoantigen has an HLA type that is compatible with, or matches type, or type and subtype, of said at least one HLA type of said patient.

11. The method of claim 1, wherein said composition in b) further comprises an expression vector, and wherein said nucleic acid sequence is part of said expression vector.

12. The method of claim 1, wherein cancer comprises Ewing's sarcoma, or Alveolar Rhabdomyosarcoma, or Paraganglioma or Synovial sarcoma.

13. The method of claim 1, further comprising treating said patient with an immune-checkpoint inhibitor.

14. The method of claim 1, wherein said a nucleic acid sequence comprises an engineered polynucleotide encoding at least a portion of said at least one fusion neoantigen, wherein said engineered polynucleotide is at least partially optimized for enhanced expression, productive co-translational protein folding, increased stability, or a combination thereof.

15. The method of claim 14, wherein at least one of the following applies:
i) wherein said polynucleotide comprises RNA, and optionally wherein said RNA is partially or fully human codon optimized;
ii) wherein said polynucleotide further comprises or encodes: a 5′ untranslated region (UTR), a 5′ cap, a 3′ UTR, a 3′ tailing sequence, or any combination thereof;
iii) wherein the polynucleotide comprises at least one chemically modified nucleotide, which is optionally a modified uracil;
iv) wherein at least 60% of the uracil in the polynucleotide encoding are chemically modified;
v) wherein the at least one chemically modified nucleotide comprises 5-methylcytosine or N1-methylpseudouridine (m1Ψ);
vi) wherein the polynucleotide is provided by a delivery vehicle, and optionally wherein the said delivery vehicle comprises a lipid nanoparticle encapsulating the polynucleotide, and further optionally wherein the lipid nanoparticle comprises a cationic lipid, a neutral and/or non-cationic lipid, a sterol, or any combination thereof, and further optionally wherein the non-cationic lipid comprises a phospholipid and/or wherein the sterol comprises cholesterol or a modification or ester thereof and/or wherein the lipid nanoparticle comprises a polyethylene glycol (PEG)-lipid conjugate.

16. A composition comprising:
a) at least a portion of at least one fusion neoantigen selected from: i) an EWSR1-FLi1 fusion protein, ii) an EWS-FEV fusion protein; iii) an EWSR1-ERG fusion protein, iv) an EWS-ATF fusion protein; v) an EWS-WT1 fusion protein; vi) a KMT2A c2318 dupC fusion protein; vii) a PAX7-FOXO1 fusion protein; viii) a CCNB3-BCOR fusion protein; ix) CIC-DVX fusion protein; x) SS18-SSX2 fusion protein; xi) a FN1fs p.F1778 fusion protein; xii) 1 ATRX pD20GG fusion protein; and xiii) frameshift 2:2FHX3 p.F297 fusion protein,
wherein said at least one fusion neoantigen is purified and isolated;
b) a nucleic acid vector comprising a nucleic acid sequence encoding at least a portion of said at least one fusion neoantigen;
c) an isolated and purified antibody, or antigen-binding fragment of said antibody, the specifically binds at least a portion of said at least one fusion neoantigen;
d) an antigen presenting cell (APC) that comprises at least a portion of said at least one fusion neoantigen,
e) a chimeric antigen receptor T-cell (CAR T cell) that expresses a chimeric antigen receptor that is specific for at least a portion of said at least one fusion neoantigen, and/or
f) an engineered polynucleotide encoding at least a portion of said at least one fusion neoantigen, wherein said engineered polynucleotide is at least partially optimized for enhanced expression, productive co-translational protein folding, increased stability, or a combination thereof.

17. The composition of claim 16, wherein said at least one fusion neoantigen is selected from any of SEQ ID NOs: 1-51, 121-170, and 179-246, and/or wherein said nucleic acid sequence encoding said at least a portion of said at least one fusion neoantigen is selected from SEQ ID NOs: 52-120.

18. The composition of claim 16, wherein said at least a portion of said at least one fusion neoantigen comprises: i) at least a portion of said EWSR1 and said FLi1 proteins; ii) at least a portion of said EWSR1 and said ERG proteins; iii) at least a portion of said FUS and said ERG proteins; or said EWSR1 and said WT1 proteins.

19. The composition of claim 16, wherein:
i) said EWS-FLi1 fusion protein is an exon 7-exon 5, 6, 7, 8, or 10 exon fusion protein;
ii) said EWS-FLi1 fusion protein is an exon 10-exon 5, 6, or 7 exon fusion protein;
iii) said EWS-FLi1 fusion protein is an exon 9-exon 8 fusion protein;
iv) said EWS-FLi1 fusion protein is an intron 10-intron 4 fusion protein;
v) said EWS-FEW fusion protein is an exon 7-exon 2 fusion protein;
vi) said EWS-ERG fusion protein is an exon 7-exon 8 or 9 fusion protein;
vii) said EWS-ERG fusion protein is an exon 10-exon 9 fusion protein;
viii) said EWS-ERG fusion protein is an exon 9-exon 7 fusion protein;
ix) said EWS-ATF fusion protein is an exon 8-exon 4 fusion protein;
x) said EWS-WT1 fusion protein is an exon 7-exon 7 or 8 fusion protein;
xi) said EWS-WT1 fusion protein is an exon 9-exon 7 fusion protein;
xii) said CIC-DVX fusion protein is an exon 20-exon 1 fusion protein; and/or
xiii) said SS18-SSX2 fusion protein is an exon 10-exon 6 fusion protein.

20. The composition of claim 16, wherein said antibody, and said antigen-binding fragment of said antibody, specifically bind said at least a portion of said at least one fusion neoantigen when it is in a complex with a major histocompatibility complex molecule.

21-27. (canceled)
